(19) United States
(12) Patent Application Publication
(10) Pub. No.: US 2026/0041929 A1
(43) Pub. Date: Feb. 12, 2026

(54) MODULAR COMBINATION MASK DEVICE FOR FACIAL DIAGNOSIS AND TREATMENT

(71) Applicant: L'Oreal, Paris (FR)

(72) Inventor: Gregoire Charraud, Jersey City, NJ (US)

(73) Assignee: L'Oreal, Paris (FR)

(21) Appl. No.: 19/291,214

(22) Filed: Aug. 5, 2025

Related U.S. Application Data
(60) Provisional application No. 63/682,173, filed on Aug. 12, 2024.

(30) Foreign Application Priority Data
Oct. 14, 2024 (FR) 2411061

(51) Int. Cl. A61N 5/06; A45D 44/00; A61F 7/00; A61M 35/00; A61N 1/32; A61N 1/40; A61N 1/44; A61N 7/00; A61N 7/02

(52) U.S. Cl. CPC A61N 5/0616; A45D 44/002; A61F 7/007; A61M 35/10; A61N 1/325; A61N 1/327; A61N 1/328; A61N 1/403; A61N 1/44; A61N 7/02; A45D 2044/007; A45D 2200/15; A45D 2200/205; A45D 2200/207; A61F 2007/0003; A61F 2007/0008; A61F 2007/0075; A61N 2005/0647; A61N 2005/0651; A61N 2005/0661; A61N 2007/0034

(57) ABSTRACT

Wearable modular mask systems for combination face and skincare diagnostics and therapeutics. Systems include devices that can be attached to wearable masks for diagnosis and treatment of skin conditions, diseases, and disorders, as well as for cosmetic improvements to the skin of the face and scalp.

### CROSS REFERENCES TO RELATED APPLICATIONS

This application claims the benefit of, and priority to, U.S. Provisional Patent Application No. 63/682,173 filed Aug. 12, 2024, and French Patent Application No. FR 2411061 filed Oct. 14, 2024; the disclosures of which are incorporated by reference herein in their entirety for all purposes.

### SUMMARY

In an aspect, the disclosure provides a modular mask system configured for cosmetic analysis and treatment, the modular mask system comprising: a wearable modular mask, comprising: a front portion attached to a rear portion that comprises a grid system configured for a cosmetic analysis, a cosmetic treatment, or both; a jig configured to accept and guide a printer device for application of a cosmetic style to a portion of skin of the individual that is adjacent to the jig; and an attachment site configured to accept and position a therapy device for application of a cosmetic treatment to a portion of skin of the individual that is adjacent to the attachment site.

In embodiments, the modular mask system further comprises the printer device.

In embodiments, the modular mask system further comprises the therapy device.

In embodiments, the modular mask system further comprises control circuitry configured to control one or more cosmetic analyses and/or treatments.

In embodiments, the modular mask system further comprises a computational device comprising circuitry configured to interact with the control circuitry for coordination, observation, or management of cosmetic analysis and treatment by the computational device.

In embodiments, the computational device comprises a smart phone, a tablet, a laptop computer, a desktop computer, a smart watch, a wearable computational device, or any combination thereof.

In embodiments, the wearable modular mask comprises a lower detachment point at which an eye portion of the wearable modular mask is detachable from a mouth portion of the wearable modular mask.

In embodiments, the modular mask system further comprises a scalp portion of the wearable modular mask, wherein a rear portion of the scalp portion comprises a grid system configured for a cosmetic analysis, a cosmetic treatment, or both.

In embodiments, the wearable modular mask comprises an upper detachment point at which the scalp portion of the wearable modular mask is detachable from an eye portion of the wearable modular mask.

In embodiments, the modular mask system comprises circuitry configured for a cosmetic analysis of a portion of skin of the individual based on a feature of the portion of skin.

In embodiments, the modular mask system comprises circuitry configured to interact with a computational device that comprises circuitry configured for a cosmetic analysis of a portion of skin of the individual based on a feature of the portion of skin.

In embodiments, the grid system is configured for an optical characterization of a portion of skin of the individual, an electrical characterization of a portion of skin of the individual, a light therapy, a micro-current treatment, a radio-frequency (RF) warming treatment, a cold plasma treatment, an acoustic energy treatment, or any combination thereof.

In embodiments, the printer device is configured for precise application of a makeup to an eyebrow portion of the face of the individual.

In embodiments, the modular mask system further comprises an aperture configured to accept at least part of a smartphone camera lens thereto for smartphone-facilitated imagery of at least a portion of the face of the individual with placement of the wearable modular mask thereto via camera circuitry of the smartphone.

In embodiments, the attachment site is positioned at a cheek portion of the wearable modular mask.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1A shows a front view of an example first wearable modular mask system, according to aspects of the disclosure.

FIG. 1B shows a rear view of the example first wearable modular mask system, according to aspects of the disclosure.

FIG. 2A shows a front view of the example first wearable modular mask system with an eye portion detached from a mouth portion, according to aspects of the disclosure.

FIG. 2B shows a rear view of the example first wearable modular mask system with the eye portion detached from the mouth portion, according to aspects of the disclosure.

FIG. 3A shows a front view of an example second wearable modular mask system, according to aspects of the disclosure.

FIG. 3B shows a rear view of the example second wearable modular mask system, according to aspects of the disclosure.

FIG. 4A shows a front view of the example second wearable modular mask system with the eye portion detached from the mouth portion and a scalp portion detached from the eye portion, according to aspects of the disclosure.

FIG. 4B shows a rear view of the example second wearable modular mask system with the eye portion detached from the mouth portion and the scalp portion detached from the eye portion, according to aspects of the disclosure.

FIG. 5 shows a schematic of an example wearable modular mask system, including example device attachments configured for at least light therapy, micro current treatment, radiofrequency (RF) electrical current warming treatment, cold plasma treatment, acoustic energy treatment, or any combination thereof, according to aspects of the disclosure.

FIG. 6 shows a schematic of an example wearable modular mask system configured for operable interaction with a smart device, such as a smart phone, according to aspects of the disclosure.

FIG. 7A shows a lower front side perspective view of an example printer device for application of a cosmetic style to a portion of a skin of an individual.

FIG. 7B shows an upper rear side perspective view of the example printer device.

FIG. 8A shows an upper exploded view of the example printer device.

FIG. 8B shows a lower exploded view of the example printer device.

FIG. 9 shows a perspective view of an example applicator capsule, according to aspects of the disclosure.

FIG. 10 shows a cross-sectional view of an example applicator capsule, according to aspects of the disclosure.

FIG. 11 shows a cross-sectional view of an example applicator capsule, according to aspects of the disclosure.

FIG. 12 shows an example formula pod and system, according to aspects of the disclosure.

FIG. 13 shows a perspective view of an example third wearable modular mask system, according to aspects of the disclosure.

FIG. 14 shows a front view of an example fourth wearable modular mask system worn by an individual, according to aspects of the disclosure.

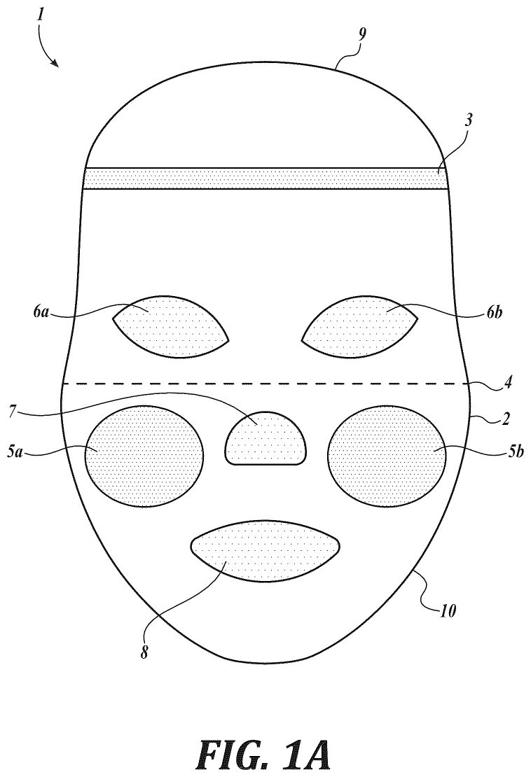

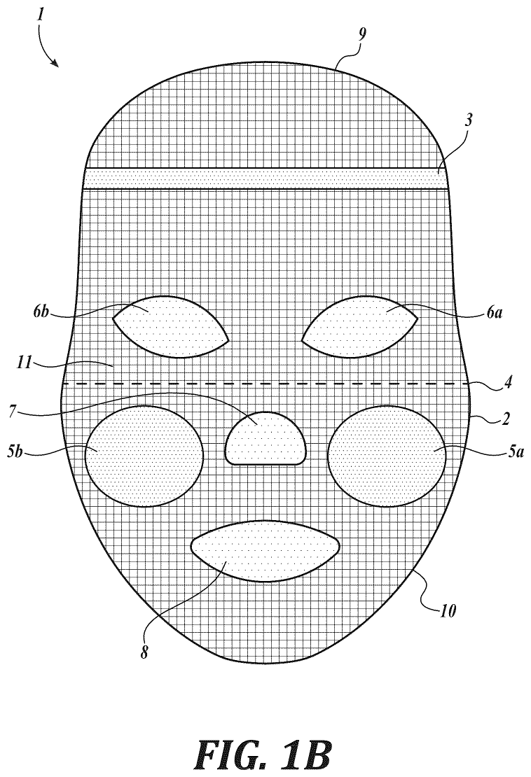

TABLE 1
Drawings elements and descriptions (FIGS. 1A-6, 13, and 14).

| Numeric reference | Description |
|---|---|
| 1 | Modular mask system |
| 2 | Wearable modular mask |
| 3 | Jig |
| 4 | Lower detachment point |
| 4a | Eye portion lower attachment point |
| 4b | Mouth portion upper attachment point |
| 5a | Attachment site (right) |
| 5b | Attachment site (left) |
| 5c | Attachment site (right) |
| 5d | Attachment site (right) |
| 5e | Attachment site (left) |
| 5f | Attachment site (left) |
| 5g | Attachment site (right) |
| 5h | Attachment site (left) |
| 5i | Attachment site (center) |
| 6a | Eye aperture (right) |
| 6b | Eye aperture (left) |
| 7 | Nose aperture |
| 8 | Mouth aperture |
| 9 | Eye portion |
| 10 | Mouth portion |
| 11 | Grid system |
| 12 | Upper detachment point |
| 12a | Scalp portion lower attachment point |
| 12b | Eye portion upper attachment point |
| 13 | Scalp portion |
| 14a | Therapy device (right) |
| 14b | Therapy device (left) |
| 15 | Printer device |
| 16a | Light therapy |
| 16b | Micro current treatment |
| 16c | Radiofrequency (RF) warming treatment |
| 16d | Cold plasma treatment |
| 16e | Acoustic therapy |
| 17 | Smart device |
| 18 | Modular mask system |
| 19 | Modular mask system |
| 20 | Securement strap |
| 21 | Power cord |

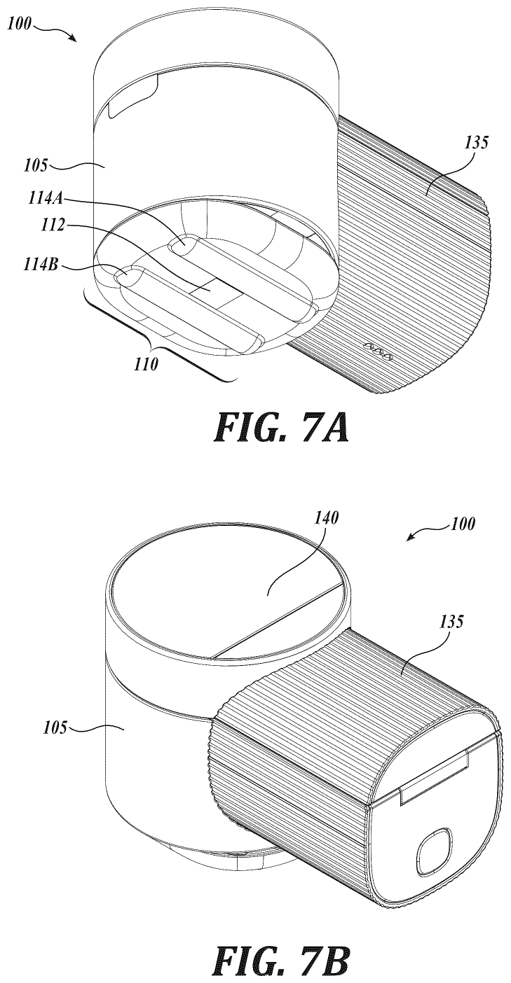

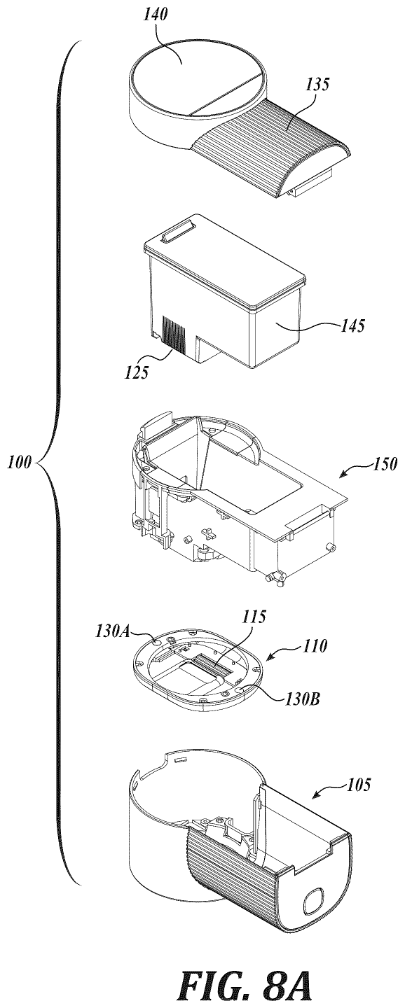

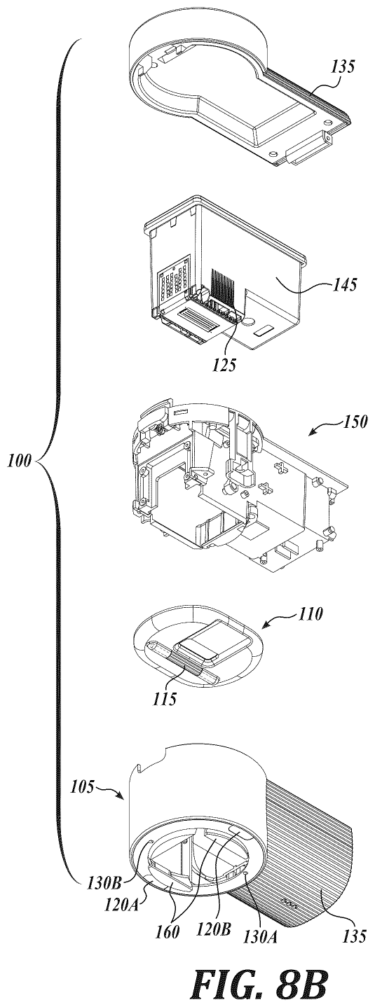

TABLE 2
Drawings elements and descriptions (FIGS. 7A-8B).

| Numeric reference | Description |
|---|---|
| 100 | Printer device |
| 105 | Housing |
| 110 | Printer |
| 112 | Printer applicator |
| 114A | Spacer |
| 114B | Spacer |
| 115 | Position sensor |
| 120A | Camera |
| 120B | Camera |
| 125 | Processor |
| 130A | Light source |
| 130B | Light source |
| 135 | Handle |
| 140 | Display |
| 145 | Reservoir |
| 150 | Internal component |
| 160 | Flexible connector |

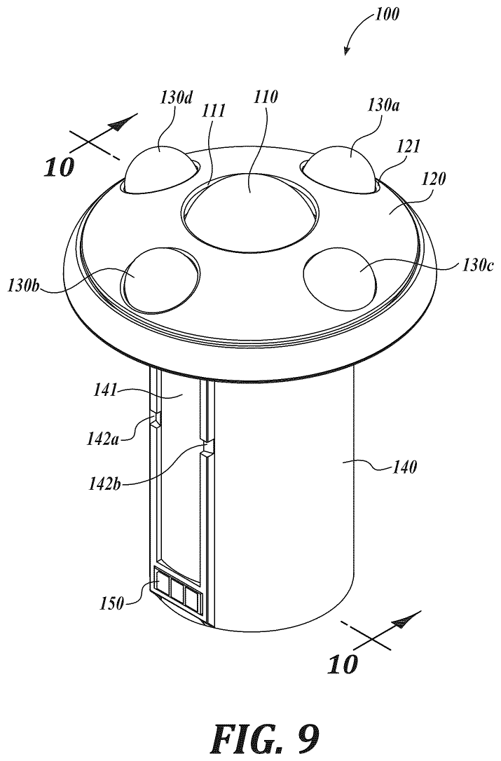

TABLE 3
Drawings elements and descriptions (FIGS. 9-12).

| Numeric reference | Description |
|---|---|
| 100 | Applicator capsule |
| 110 | Roller ball |
| 111 | Roller ball aperture |
| 112 | Formula pod |
| 113 | Formula pod microchip |
| 120 | Contact face |
| 121 | Electrode aperture |
| 130a | Electrode |
| 130b | Electrode |
| 130c | Electrode |
| 130d | Electrode |
| 140 | Insert |
| 141 | Adapter |
| 142a | Locking groove |
| 142b | Locking groove |
| 150 | Electrode contact |
| 163 | Adapter |
| 161a | Aperture (outer portion) |
| 161b | Aperture (inner portion) |
| 162 | Interior |
| 200 | Body |
| 201 | Insert attachment |
| 203 | Reservoir attachment |
| 204 | Reservoir |
| 207 | Aperture |
| 208 | Electrical connection |
| 900 | Microcurrent system |

The foregoing aspects and many of the attendant advantages of this disclosure will become more readily appreciated as the same become better understood by reference to the following detailed description, when taken in conjunction with the accompanying drawings.

### DETAILED DESCRIPTION

Consumers of cosmetic devices, including personal care devices for face, body, head, skin, or the like, diagnostic devices, treatment devices, or the like, typically own multiple devices that each meets only a subset of the total skincare or personal care needs of the individual. The individual consumer therefore typically purchases, owns, and manages multiple devices, throughout a lifetime of maintenance, repairs, and replacement of consumables. Moreover, some cosmetic devices are difficult to operate precisely and consistently, causing user frustration.

Accordingly, the disclosed technologies and methodologies are directed to providing a personal care device platform configured to integrate diverse cosmetic diagnostics and treatments, to enhance convenience, ease, accuracy, and consistency, and other long-felt and unmet needs in the art.

### Modular Mask Systems

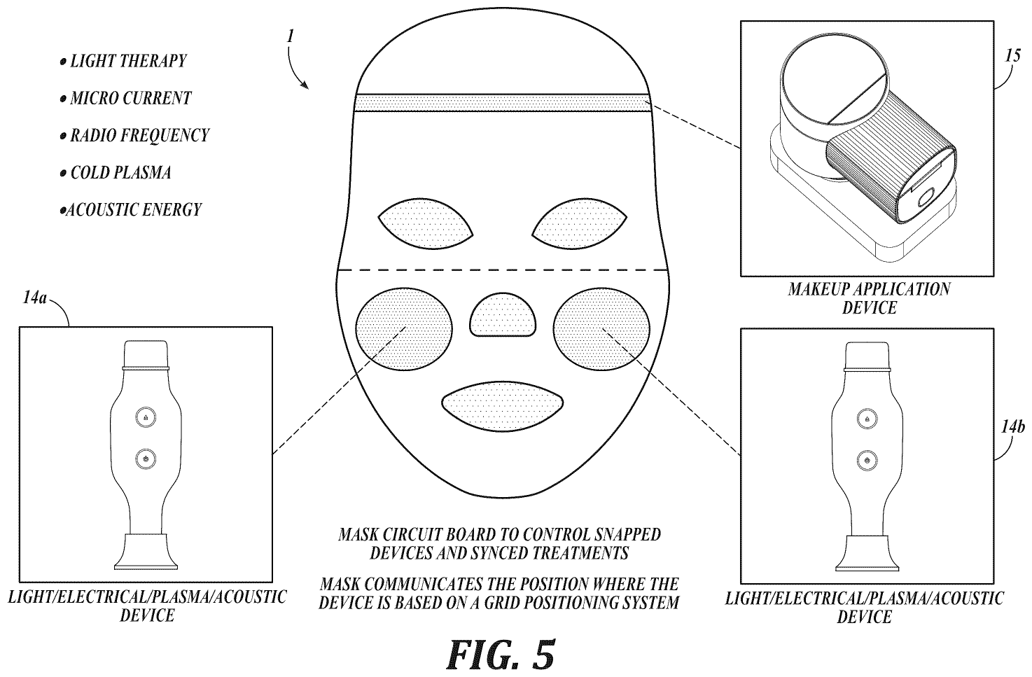

Referring to FIG. 5, an example wearable modular mask system 1 includes or is configured to be compatible with various example device attachments (e.g., 14a, 14b, 15). Non-limiting examples of device attachments (e.g., 14a, 14b, 15) include modular components configured to deliver light therapy, micro current treatment, radiofrequency (RF) electrical current warming treatment, cold plasma treatment, acoustic energy treatment, or any combination thereof, according to aspects of the disclosure. In an embodiment, the devices are configured to provide one or more diagnostic and/or treatment modalities. In an embodiment, the device is reversibly or temporarily attached to the wearable mask, which can hold the devices in a relatively fixed, controlled, or limited position relative to the user's face for consistent and reliable diagnostics and treatments. Since the positions of the devices relative to the face of the individual are more consistent or reproducible, the devices may not necessarily need to make as many physical adjustments in positioning for diagnostics and/or treatments, and the processing power needed for diagnostics and/or treatments can be reduced leading to a more efficient system.

The disclosure provides modular mask systems that are configured for use by consumers at home or in their own space, without necessarily requiring a visit to a clinic or spa. In an embodiment, the system is configured to visually map a user's face (e.g., generation of a profile of an individual) and identify treatment areas. In an embodiment, a device is configured to “snap-in” to a mask of the system for use, for example, a snap-in skin printing module, a snap-in LED treatment module, and the like. For an LED treatment, an LED light therapy device can be plugged into the mask and connected with a plurality of waveguides that deliver light to the correct places. The LED light does not need to be moved around; it stays static, relative to the user's face, and the light is delivered through the waveguides (which can be arranged as a grid system, as described herein) for delivery of the light to all or many portions of the skin of the face and/or scalp of the user. Since the wearable mask includes holes for the eyes, and optionally, holes for the mouth and/or nose, the mask can be worn in daily life with minimal interruption. In embodiments, the mask system can have a Peltier heating and/or cooling system (e.g., warmth, cooling, or both). In embodiments, the mask system can be configured for facilitation of an interaction with a virtual realty (VR) and/or augmented reality (AR) system or device by a user of the mask system when worn by the user.

As shown by way of non-limiting examples at FIGS. 1A-4B, in various aspects, the disclosure provides a modular mask system 1 configured for cosmetic analysis and treatment, the modular mask system 1 comprising: a wearable modular mask 2, comprising: a front portion attached to a rear portion that comprises a grid system 11 configured for a cosmetic analysis, a cosmetic treatment, or both; a jig 3 configured to accept and guide a printer device (e.g., 100 of FIGS. 7A-8B) for application of a cosmetic style to a portion of skin of the individual that is adjacent to the jig 3; and an attachment site configured to accept and position a therapy device (e.g., 100 and/or 900 of FIGS. 9-12) for application of a cosmetic treatment to a portion of skin of the individual that is adjacent to the attachment site.

In embodiments, the modular mask system 1 further comprises control circuitry configured to control one or more cosmetic analyses and/or treatments. Control circuitry can comprise dedicated hardware circuitry, processor circuitry (e.g., circuitry including a non-transitory machine-readable storage medium having stored thereon instructions which, when executed by a processor, cause or configure the processor to perform all or part of a method or operation for control of one or more cosmetic analyses and/or treatments; e.g., including software, firmware, or the like). In embodiments, the modular mask system 1 does not include control circuitry, but includes substantially a wearable modular mask 2, as a passive physical platform, that holds one or more modular devices with their own control circuitry, as described herein.

In embodiments, the modular mask system 1 further comprises a computational device comprising circuitry configured to interact with the control circuitry for coordination, observation, or management of cosmetic analysis and treatment by the computational device. In non-limiting example embodiments, the computational device comprises a smart phone, a tablet, a laptop computer, a desktop computer, a smart watch, a wearable computational device, or any combination thereof. In embodiments, the modular mask system 1 comprises circuitry configured for a cosmetic analysis of a portion of skin of the individual based on a feature of the portion of skin. In embodiments, the modular mask system comprises circuitry configured to interact with a computational device that comprises circuitry configured for a cosmetic analysis of a portion of skin of the individual based on a feature of the portion of skin.

In embodiments, the modular mask system 1 can be configured for generation, acceptance, management, and/or editing of one or more user profiles, which can be particular to an individual. The user profile can enable the system to configure itself for a particular set of one or more diagnostics and/or treatments particular to the individual. The user profile can include information such as skin type or color, locations of permanent scarring, favorited or previously used diagnostics and treatments, and the like.

A grid system 11, positioned on a rear portion of the wearable mask, can be comprised of one or more structures configured for one or more diagnostics, therapies, or treatments, as described herein. In embodiments, the grid system 11 is configured for an optical characterization of a portion of skin of the individual (e.g., through one or more waveguides configured for capture of light of a portion of skin of the individual), an electrical characterization of a portion of skin of the individual (e.g., through one or more capacitance measurements of current or resistance for detection of scarring, moisture levels, or the like, with use of one or two or more electrical contact pins for capacitance measurements), a light therapy (e.g., through light emitting diodes (LEDs) that transmit light through a plurality of waveguides arranged as a grid), a micro-current treatment (e.g., through a micro-current therapy device), a radio-frequency (RF) warming treatment (e.g., through a RF warming therapy device), a cold plasma treatment (e.g., through a cold plasma therapy device), an acoustic energy treatment (e.g., through an acoustic energy therapy device), or any combination thereof.

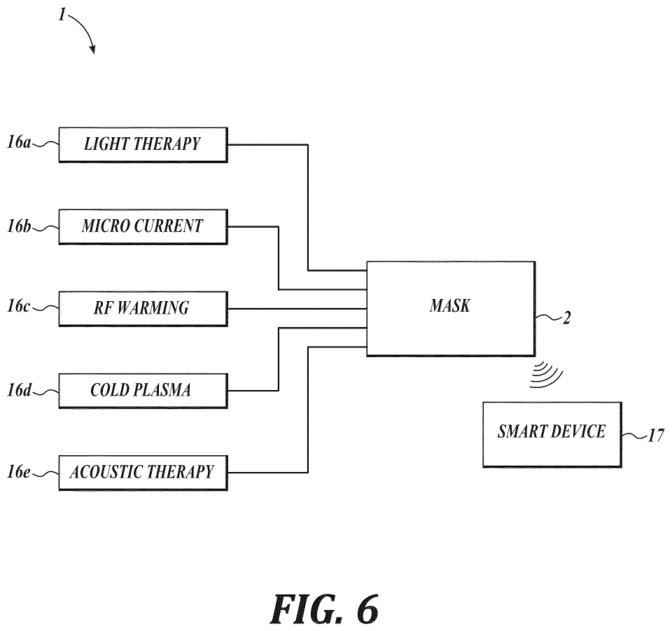

In various embodiments, a modular mask system is configured for operable interaction between the mask 2 or another element of the system and a smart device 17, such as a smart phone, as shown by way of a non-limiting example at FIG. 6. A mask system 1 can include various device attachments (16a, 16b, 16c, 16d, 16e) configured for at least light therapy, micro current treatment, radiofrequency (RF) electrical current warming treatment, cold plasma treatment, acoustic energy treatment, or any combination thereof, according to aspects of the disclosure. In embodiments, the modular mask system further comprises an aperture configured to accept at least part of a smartphone camera lens thereto for smartphone-facilitated imagery of at least a portion of the face of the individual with placement of the wearable modular mask thereto via camera circuitry of the smartphone.

### Wearable Modular Masks

In various aspects, the disclosure provides wearable, modular masks, configured as form factors that are comfortable when worn on the head and functional for cosmetic analyses and treatments as described herein. In embodiments, the wearable modular mask is configured as a modular platform with which multiple different cosmetic diagnostics and/or treatments can be combined and be operable together or individually. Wearable modular masks can be generally shaped or configured to attach to and/or cover at least part of an individual's face and/or head, e.g., the scalp, the eye and/or forehead area, the nose and/or cheek area, the mouth and/or chin area, and the like, or any combination thereof.

In embodiments, one or more devices (e.g., printer devices, therapy devices, and the like) is modular and removably attachable to an attachment site of the modular mask system for use, in various aspects of the disclosure. In embodiments, an attachment site is positioned at a cheek portion of the wearable modular mask. For example, a therapy device can be attached to the wearable modular mask by way of a snap connection with attachment site 5a and/or 5b of the wearable modular mask of any of FIGS. 1A-4B.

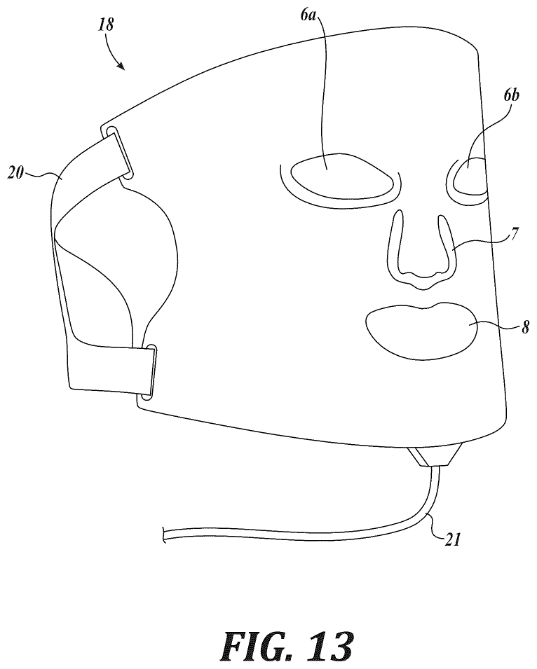

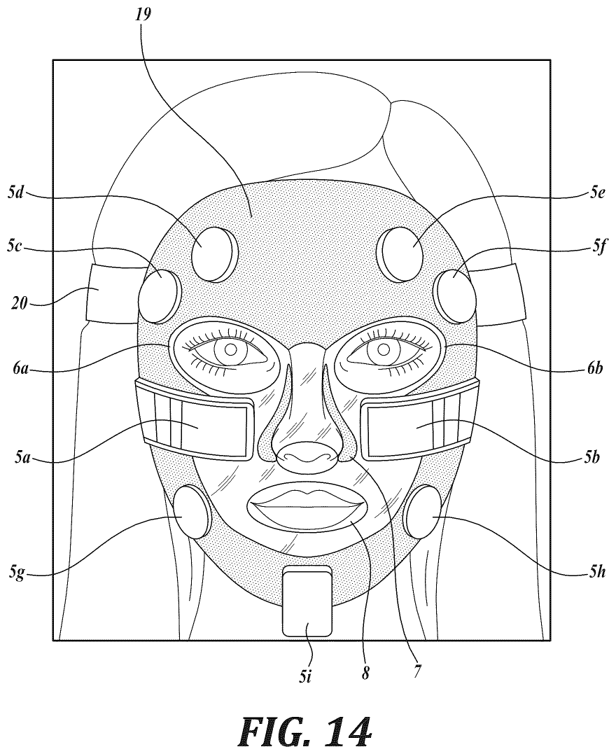

As a further non-limiting example, in embodiments, a therapy and/or diagnostic device is configured to be attached to a wearable modular mask system 18 of FIG. 13 by way of a snap connection with an attachment site (e.g., attachment site 5a, attachment site 5b, or any combination thereof). As a further non-limiting example, a therapy and/or diagnostic device can be attached to a wearable modular mask system 19 of FIG. 14 by way of a snap connection with an attachment site (e.g., attachment site 5a, attachment site 5b, attachment site 5c, attachment site 5d, attachment site 5e, attachment site 5f, attachment site 5g, attachment site 5h, attachment site 5i, or any combination thereof).

As a further non-limiting example, as shown at FIGS. 1A-4B, a printer device can be attached to a jig 3 of the mask. The jig 3 can comprise a guide rail that enables the printer device to move from left to right and from right to left along the guide rail, e.g., substantially parallel with the shape of the jig 3, such that the printer device can consistently apply a cosmetic style to the subject's face or eyebrows. Since the printer device moves along the jig 3, which maintains a constant vertical position of the printer device, the printer device has little or no deviations from a substantially constant horizontal movement and the printer device can require little or no corrections in nozzle placement for an accurate and precise application of the cosmetic style.

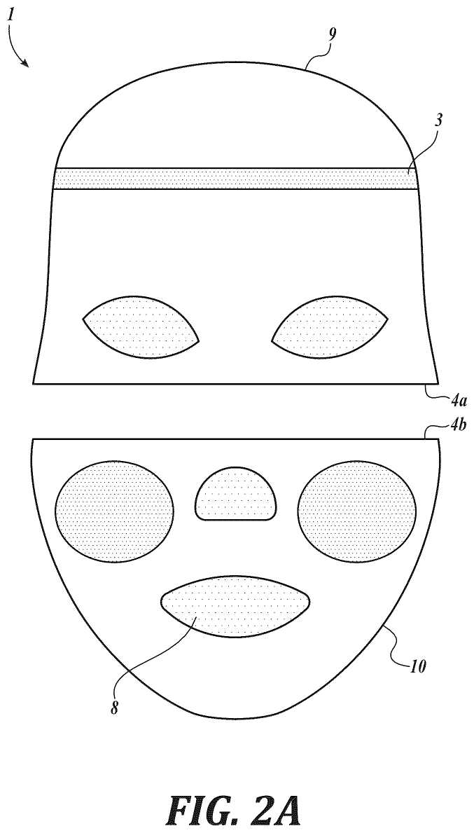

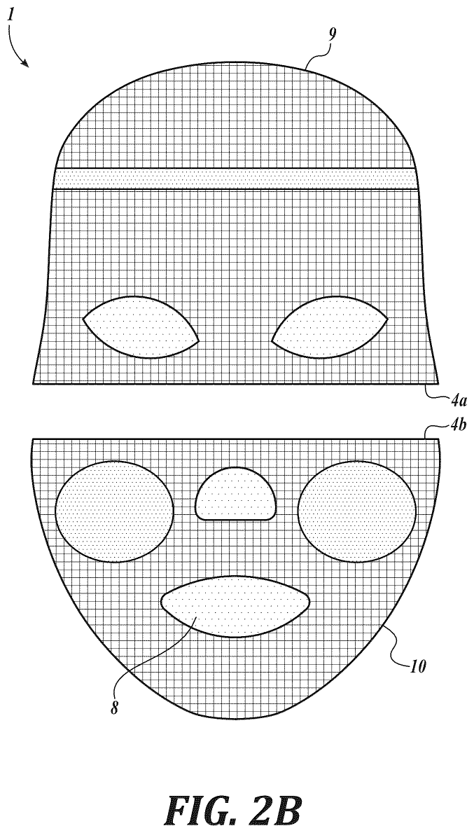

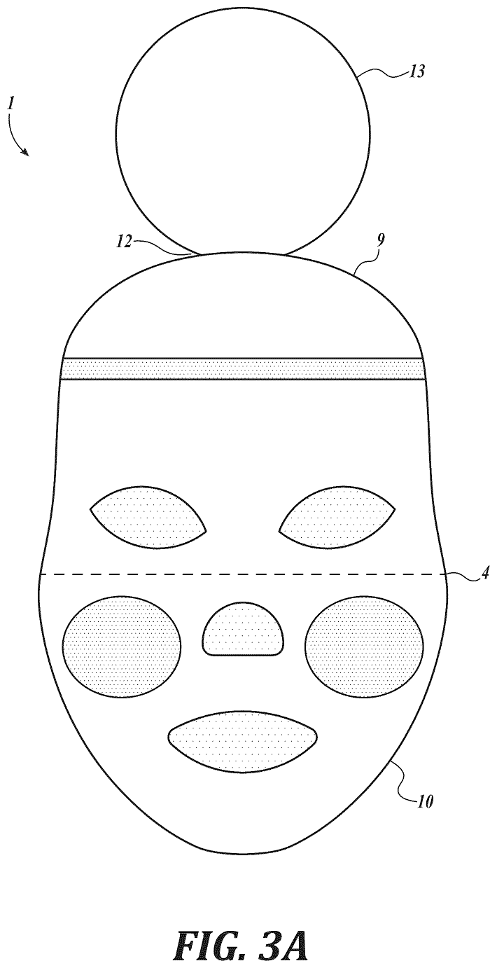

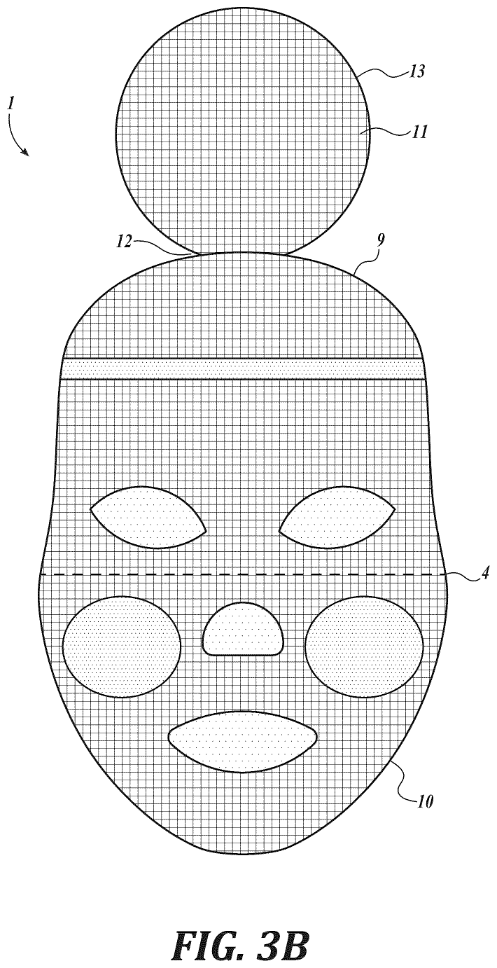

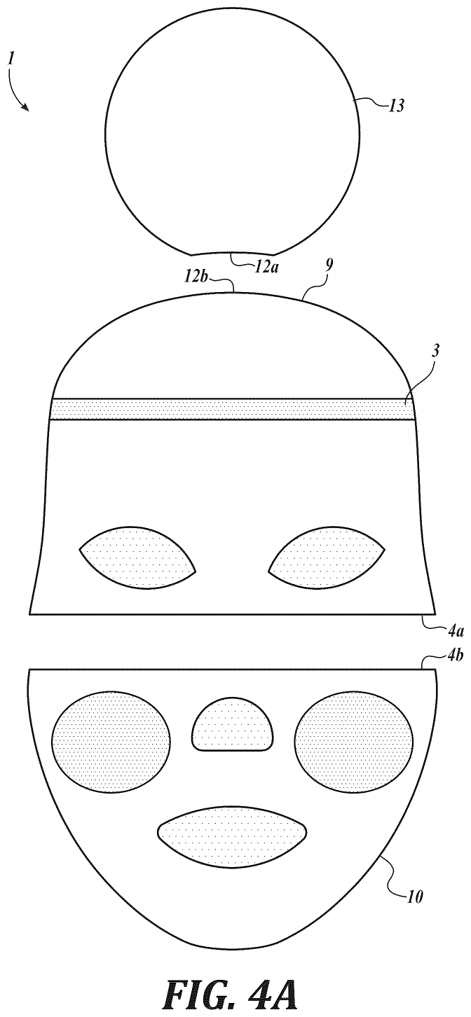

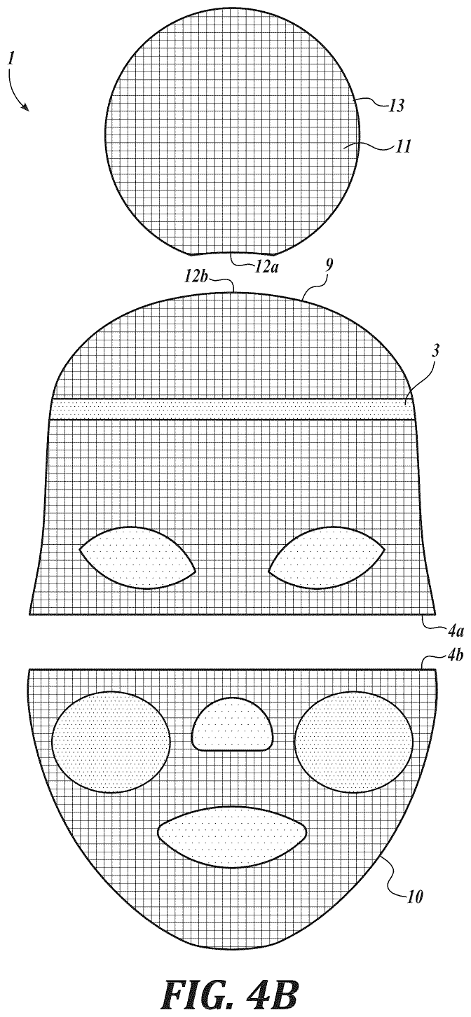

In embodiments, a wearable modular mask comprises a lower detachment point (e.g., 4 of FIGS. 1A, 1B, 3A, and 3B), at which an eye portion (e.g., 9 of FIGS. 1A, 1B, 3A, and 3B) of the wearable modular mask is detachable from a mouth portion (e.g., 10 of FIGS. 1A-4B) of the wearable modular mask, as shown by way of non-limiting examples at FIGS. 2A, 2B, 4A, and 4B. In this manner, a user can use all or part of the mask for one or more skincare diagnostics and/or treatments. In embodiments, the modular mask system further comprises a scalp portion (e.g., 13 of FIGS. 3A, 3B, 4A, and 4B) of the wearable modular mask, wherein a rear portion of the scalp portion 13 comprises a grid system 11 configured for a cosmetic analysis, a cosmetic treatment, or both. In embodiments, the wearable modular mask comprises an upper detachment point at which the scalp portion of the wearable modular mask is detachable from an eye portion of the wearable modular mask, as shown by way of non-limiting examples at FIGS. 4A and 4B.

Non-limiting examples of form factors that can be implemented for a wearable modular mask of the disclosure are shown at FIGS. 1A-4B, 13, and 14. A wearable modular mask (1, 18, 19) can comprise a shape and curvature that is stylish and configured to be fitted to a wearer's face and/or scalp. As shown at FIG. 13, a wearable modular mask 18 can comprise a right eye aperture 6a, a left eye aperture 6b, a nose aperture 7, and a mouth aperture 8. The wearable modular mask 18 can comprise a securement strap 20, such as an elastic or adjustable headband, for securement of the wearable modular mask 18 to the wearer's head. In various embodiments, including as shown at FIG. 13, the wearable modular mask 18 can comprise an electrical power circuit that comprises a power cord 21 that is configured to be plugged into an alternating current (A/C) outlet, for example. The electrical power circuit can be used to power or recharge one or more devices (e.g., printer devices, therapy devices, and the like) attached or attachable to the wearable modular mask 18.

As shown at FIG. 14, a wearable modular mask 19 can comprise a right eye aperture 6a, a left eye aperture 6b, a nose aperture 7, and a mouth aperture 8. The wearable modular mask 19 can comprise a plurality of attachment sites (e.g., attachment site 5a, attachment site 5b, attachment site 5c, attachment site 5d, attachment site 5e, attachment site 5f, attachment site 5g, attachment site 5h, and attachment site 5i) for securement of a printer device, a therapy device, and/or a diagnostic device to the wearable modular mask 19 for use, in embodiments. In embodiments, the wearable modular mask 19 can comprise a securement strap 20, such as an elastic or adjustable headband, for securement of the wearable modular mask 19 to the wearer's head.

### Printer Devices

In embodiments, a modular mask system further comprises a printer device, for example, all or one or more portions of a printer device as described in U.S. patent application Ser. No. 18/345,339, the contents of which are incorporated by reference herein in their entirety for all purposes. In embodiments, the printer device is configured for precise application of a makeup to an eyebrow portion of the face of the individual.

As used herein, the term “printer device” includes, but is not necessarily limited to, any device that is configured for an application of a make-up or other cosmetic composition onto a portion of skin of an individual and includes, but is not necessarily limited to, devices that operate by printing, spraying, projecting, directly applying through physical contact and transfer, micro air spraying, pigment projecting, and the like.

FIGS. 7A-8B show several views of an example printer device for application of a cosmetic style to a portion of a skin of an individual. Printer devices, systems, and methods can be implemented for autonomous, semi-autonomous, and assisted manual application of a cosmetic style to a portion of skin of an individual, for example, by attachment of a printer device to a jig (as described herein) such that the printer device travels along the jig to reliably apply a cosmetic style to an individual. The disclosed approaches enable the individual to correct application of the composition in real time for more accurate placement of the cosmetic style to the portion of skin. Printer devices are useful alone or in combination with smart devices, such as smartphones, for planning, selection, and implementation of cosmetic styles among a plurality of cosmetic styles.

As shown at FIG. 7A and FIG. 7B, a printer device 100 includes a printer applicator 112, a position sensor, and a reservoir for compositions for the cosmetic style. A display 140 of the printer device 100, if included in an embodiment, can represent the portion of skin as a plurality of guide segments, and the printer applicator as a visual indicator relative to the plurality of guide segments based on the position sensor. The depiction of the visual indicator responds to changes in the position of the printer applicator relative to the portion of the skin to visually guide the individual to accurately apply the cosmetic style, and a feedback device or component alerts the individual if the application of the cosmetic style deviates or starts to deviate from the portion of the skin to enable the individual to correct the course of the application. A user can use these or other elements to register the printer device 100 with a position of the user's eyebrows, such that attachment to and travel along the jig of the wearable modular mask by the printer device 100 enables a consistent application of cosmetic styles.

A printer device 100 is configured for application of a cosmetic style to a portion of a skin of an individual, and comprises a printer 110, a position sensor, a display 140, and circuitry for carrying out all or part of an operation or method of the disclosure. The printer 110 comprises a printer applicator 112 operably connected to a reservoir (145 of FIG. 8A) comprising a dye therein, and the position sensor is configured to detect a position of the printer applicator 112 relative to the portion of the skin. The display 140 can be configured to represent the portion of the skin as a plurality of guide segments, and represent the position of the printer applicator as a visual indicator (e.g., an arrow, a circle, a square, a triangle, and the like) relative to the plurality of guide segments. A position of the visual indicator as depicted by the display 140 responds to a change in the position of the printer applicator 112 relative to the portion of the skin, as a result of the position sensor. Positional data generated during an application of the cosmetic style can be evaluated as part of a quality control process, for example.

Circuitry of printer device 100, which includes but is not limited to a processor, a microprocessor, processor circuitry, and/or dedicated hardware circuitry, operably connects the printer 110, the position sensor, and the display 140. The circuitry is configured to direct the printer 110 to print the cosmetic style with passage of the dye from the reservoir through the printer applicator 112 to the portion of the skin, compute the position of the printer applicator 112 relative to the portion of the skin based on the position sensor, compute a depiction of the visual indicator relative to a guide segment of the plurality of guide segments based on the position of the printer applicator relative to the portion of the skin, and transmit to the display 140 for the depiction of the visual indicator relative to the plurality of guide segments by the display 140. In embodiments, circuitry of printer device 100 is configurable with a processor and processor-executable instructions stored on a non-transitory machine-readable medium of printer device 100, as a non-limiting example, but other approaches for configuring circuitry of printer device 100 can be implemented in embodiments.

As shown at FIGS. 7A and 7B, in embodiments, printer device 100 includes a housing 105 and a handle 135. Printer device 100 is shown with a cylindrical housing 105 and a cylindrical handle 135, but can be implemented according to any number of shapes and form factors. In embodiments, printer device 100 does not have handle 135 as shown. In embodiments, printer device 100 includes internal circuitry, including a processor, a power source, such as a battery, and the like, for electronic operation of printer device 100.

In embodiments, printer device 100 includes a processor for execution of instructions stored on a non-transitory machine-readable medium, for enabling the processor to carry out all or part of a method or process of the disclosure. In embodiments, the processor is configured to receive a makeup image file, detect a position and a curvature of a portion of skin of an individual based on the position sensor, and direct printer 110 to print a cosmetic style based on the makeup image file at a location on the portion of skin. In embodiments, the location is determined by a cosmetic style. For example, a lipstick cosmetic style can be printed on the lips of the individual, a brow makeup can be printed to the eyebrow of the individual, and the like.

In embodiments, printer device 100 is powered through a wired connection, e.g., a wired electrical connection with a source of alternating current; however, in embodiments, printer device 100 is independently powered, such as with a battery or a capacitor. In embodiments, printer device 100 includes a charging port configured to receive electricity from a power source to recharge a battery or capacitor of the printer device 100.

In embodiments, housing 105 houses printer 110. In embodiments, printer 110 is positioned on a first side of the printer device 100, and display 140 is positioned on a second side of the printer device 100, as shown at FIGS. 7A and 7B. In embodiments, printer 110 includes a printer applicator 112 and one or more spacers 114A and 114B.

In embodiments, printer applicator 112 is configured to facilitate printer 110, as shown at FIG. 8B, to print a cosmetic style onto a surface. In embodiments, printer applicator 112 is rectangular, square, circular, organically shaped, or the like. In embodiments, printer applicator 112 is in the middle of a front side of housing 105. In embodiments, printer applicator 112 is between spacers 114A, 114B.

While two spacers 114A and 114B are illustrated, it should be understood that any number and configuration of spacers 114A, 114B can be positioned on printer 110. In embodiments, spacers 114A, 114B are rounded polygons, as shown at FIG. 7A, but it should be understood that spacers 114A, 114B can be implemented as any number of forms including spherical, rectangular, and organically shaped. In embodiments, spacers 114A, 114B are configured to contact a surface while printer device 100 is passed over it, such that an optimal distance between printer 110 (or printer applicator 112) and the surface is maintained. In embodiments, spacers 114A, 114B have a thickness that enables printer applicator 112 to be in contact with a curved surface. In embodiments, spacers 114A, 114B are configured to roll. In embodiments, spacers 114A, 114B include at least one position sensor, as described herein. In embodiments, in addition to maintaining a distance between printer applicator 112 and the surface, spacers 114A, 114B are configured to roll on the surface as printer device 110 prints the cosmetic style to the surface.

In embodiments, printer device 100 includes a position sensor operably coupled to printer 110, as shown at FIG. 8A. In embodiments, position sensor is housed inside housing 105, but in embodiments, position sensor is located on the front side of printer device 100 with printer applicator 112. In embodiments, the position sensor is positioned inside one or both spacers 114A, 114B. In embodiments, printer device 100 further includes a camera, as shown at FIG. 8A. In embodiments, the camera is configured to capture a plurality of images as printer 110 moves over a portion of skin, such as a facial feature of an individual. In embodiments, the facial feature is an eyebrow, a nose, an eye, a wrinkle, acne, or the like.

In embodiments, printer 110 is a rotatably adjustable body printer 110. In embodiments, printer 110 is configured to articulate to scan a surface more accurately, such as a body, skin, or hair. In such embodiments, position sensor 115 can be a sensor wheel as described herein. In operation, position sensor 115 contacts the surface and rolls as the printer 110 scans the surface. In such embodiments, printer device 100 is able to consider the curvature of the surface, which can be a portion of a human body. In embodiments, printer 110 is adjustable to fit the needs of different body types and printing environments. In embodiments, printer 110 has an adjustable printer applicator 112. In embodiments, spacers 114A, 114B are movable or adjustable to change the size of printer applicator 112. In embodiments, printer applicator 112 is concave or convex to better contact the surface. In embodiments, printer 110 configured for being articulated, so as to better contact the surface. In embodiments, printer 110 is coupled to printer device 100 with a flexible connector 160, as shown at FIG. 8B. In embodiments, the flexible connector is a pivot, a hinge, or a joint. In embodiments, the flexible connector allows printer 110 to be articulated. In embodiments, this allows for more accurate scans of a surface. In embodiments, this further allows the printer 110 to determine a curvature of a surface.

In embodiments, printer device 100 includes a display 140, configured for use as a user interface. Though display 140 is shown on the back side of printer device 100, in embodiments, display 140 is a separate component, such as a smartphone or tablet. In embodiments, display 140 is round, but in other embodiments, can be implemented in any form, such as rectangular or oblong. In embodiments, display 140 includes one or more actuators, such as buttons or keys. In embodiments, display 140 includes a touch type capacitance button. In embodiments, display 140 is a touchscreen. In embodiments, the display includes one or more output modules configured to output an alert, such as feedback, to the user. In embodiments, the alert is a sound, vibration, or the like. In embodiments, the alert includes an indication as to how or in what direction to move printer device 100 during use.

FIG. 8A shows an upper exploded view, and FIG. 8B shows a lower exploded view, of the example printer device. In embodiments, printer device 100 includes an internal component 150, printer 110, and position sensor 115. In embodiments, printer device 100 includes a reservoir 145 and a processor 125. In embodiments, internal component 150 is configured to hold printer 110 in place within housing 105. In embodiments, internal component 150 is structurally coupled to printer 110 and reservoir 145.

In embodiments, printer 110 includes position sensor 115 and one or more cameras 120A, 120B. In embodiments, cameras 120A, 120B are located on printer 110 but in embodiments, cameras 120A, 120B are located on housing 105. In embodiments, as printer 110 moves across a surface, such as an individual's face, cameras 120A, 120B capture a plurality of images of the surface. In embodiments, cameras 120A, 120B capture a plurality of images of a facial feature as the printer device 100 moves over the facial feature. In embodiments, printer device 100 includes two cameras 120A and 120B. In embodiments, such as illustrated at FIG. 8B, a first camera 120A is located at a first portion of the printer device 100, and a second camera 120B is located at a second portion of the printer device 100, e.g., opposite the first portion.

In embodiments, as shown at FIG. 8B, printer device 100 includes one or more light sources 130A, 130B. In embodiments, light sources 130A, 130B are LEDs. Though two light sources 130A, 130B are shown, any number of light sources can be implemented on the printer device 100, according to embodiments. In embodiments, light sources 130A, 130B are positioned on the printer 110, but in other embodiments, light sources 130A, 130B are positioned on the front side of printer device 100.

In embodiments, printer 110 includes one or more position sensors 115. While one position sensor 115 is shown at FIG. 8A, it should be understood that any number of position sensors 115 can be implemented. In embodiments, at least one position sensor 115 is a rolling position sensor 115, such as a sensor wheel. In such embodiments, position sensor 115 is configured to roll across the facial feature as printer 110 is moved over the facial feature. In this manner, position sensor 115 detects a position of the facial feature as the printer device 100 moves over the facial feature. In embodiments, position sensor 115 is further configured to detect the curvature of the facial feature or the user's face, i.e., the portion of the skin of the individual.

In embodiments, printer device 100 includes a processor 125. In embodiments, processor 125 is operably and/or communicatively coupled to printer 110, position sensor 115, and one or more cameras 120a, 120b. The processor 125 is configurable to receive a makeup image file, detect a position and a curvature of the portion of the skin based on the position sensor, and direct the printer to print the cosmetic style based on the makeup file at a location. In embodiments, processor 125 is further configured to detect the lighting of the facial feature, and direct one or more light sources 130A, 130B to illuminate the facial feature. While one processor 125 is illustrated, it should be understood that any number of processors can be implemented into printer device 100.

In embodiments, printer device 100 includes a reservoir 145. In embodiments, the reservoir 145 is configured to hold one or more cosmetic inks or dyes, or other compositions for the cosmetic style. In embodiments, the reservoir holds any number of cosmetic inks or dyes as needed to print the cosmetic style. In embodiments, the reservoir 145 includes one or more cartridges, such that reservoir 145 can hold any number of colors, compositions, finishes, or formulations of the cosmetic inks or dyes.

In embodiments, processor 125 is further communicatively coupled to reservoir 145 and printer 110. In embodiments, processor 125 directs reservoir 145 and printer 110 to fabricate a cosmetic style, such as a temporary tattoo, or makeup printed in the shape of the facial feature. In embodiments, the cosmetic style is selected from an eyebrow, an eyeshadow, a concealer, a primer, a foundation, a blush, a lipliner, a lipstick, a bronzer, an eyeliner, a freckle pattern, a facial hair, a hair design, such as facial hair or a hairline design, or a highlighter.

In embodiments, printer 110 is coupled to the printer device 100 with a flexible connector 160. In embodiments, flexible connector 160 is a pivot, a hinge, or a joint. In embodiments, flexible connector 160 enables printer 110 to be articulated. In embodiments, this allows for more accurate scans or printing of the surface. In embodiments, this further allows printer 110 to determine a curvature of the surface.

### Therapy Devices

In embodiments, the modular mask system further comprises a therapy device, for example, all or a portion of a therapy device as described in U.S. patent application Ser. No. 18/756,759, the contents of which are incorporated by reference herein in their entirety for all purposes.

FIGS. 9-12 show views of an example applicator capsule and an example formula pod and system, according to aspects of the disclosure.

Iontophoresis is a non-invasive technique wherein a physiologically acceptable amount of electric current (e.g., up to about 0.5 mA/cm² or typically 10 V or less) is used to facilitate transdermal delivery of charged and/or neutral molecules. Iontophoresis does not tend to disrupt the skin barrier in promoting transdermal flux, and acts directly on one or more compounds of a composition applied to the skin to deliver the compound into deeper layers of the skin. Electroporation applies a higher voltage (typically about 100 V) pulse for a very short (microseconds to milliseconds) duration to permeabilize the skin.

These techniques can potentially enable drug delivery across the skin and expand the scope of transdermal delivery to include not only small molecules but large molecules as well, such as proteinaceous drugs of the biotechnology industry. However, the potential of these techniques has not been fully realized due to the skin maintaining a barrier that restricts or prevents passage of the molecules therethrough. In addition, iontophoresis and electroporesis systems are typically configured for use with distinct classes of molecules that are delivered with different electrical currents or programs. A therapeutic composition that includes one or more biologically active molecules that are from distinct classes may involve both iontophoresis and electroporesis systems, and this is an inconvenience for the user or clinician and can result in lower adherence to a treatment plan or further health complications due to inadequate treatment.

Accordingly, the wearable modular mask system of the disclosure can be implemented with intradermal and transdermal therapeutic delivery systems that are configured for iontophoresis and electroporesis, that can more effectively deliver a wide range of molecule types to and through the skin, and that are configured to warm compositions during use to relax the skin layers and widen the gaps between skin cells for more effective delivery of compounds therein. The present disclosure addresses these and other long-felt and unmet needs in the art.

As shown at FIGS. 9-11, an example applicator capsule 100 comprises a roller ball 110, positioned within a roller ball aperture 111, that makes a rolling contact with the skin of a subject and rolls across the skin when a contact face 120 is placed adjacent to the skin during use. In the shown embodiment, the applicator capsule 100 comprises a plurality of electrodes, including electrodes 130a, 130b, 130c, and 130d, configured for a transmission of an electrical current through the portion of skin of the subject during use. While four electrodes are included in the example embodiment, other amounts of electrodes can be implemented, in embodiments, without departing from the scope and spirit of the disclosure. Examples of alternative amounts of electrodes include but are not limited to one electrode, two electrodes, three electrodes, five electrodes, six electrodes, seven electrodes, eight electrodes, nine electrodes, ten electrodes, or more.

In the shown embodiment, the applicator capsule 100 comprises an insert 140 that is configured to be inserted into a body of the system for attachment of the applicator capsule 100 to the body. The shown insert 140 includes an adapter 141 fitted with locking grooves 142a and 142b, which can slidingly and reversibly lock to the body when the applicator capsule 100 is attached thereto. An electrode contact 150 is operably connected to the electrodes 130a, 130b, 130c, and 130d, by way of one or more electrical connections, for delivery of the electrical current from a power source through the electrode contact 150 to the electrodes 130a, 130b, 130c, and 130d. While the shown insert 140 includes a substantially spherical cross sectional shape with a flat portion for the adapter 141, other cross sectional shapes can be implemented without departing from the scope and spirit of the disclosure, with the understanding that the insert 140 would need to be able to be secured to the body of the system, and the body can optionally include a correspondingly shaped cavity for receipt of the insert 140 therein.

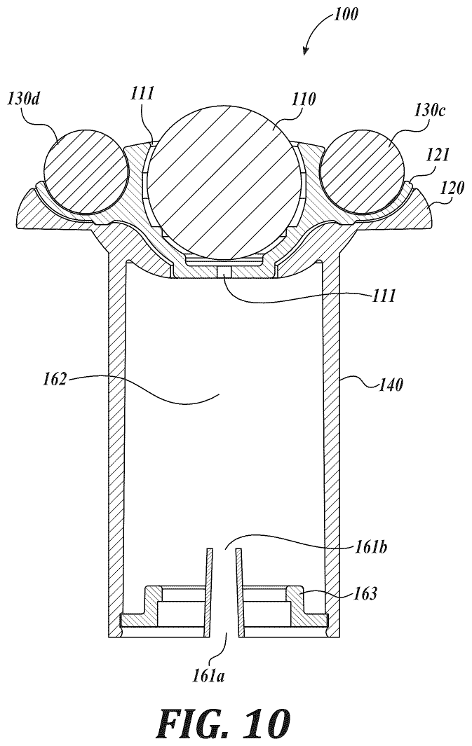

In the embodiment of FIG. 10, an interior 162 of the applicator capsule 100 is fluidly connected to an interior of the body of the system by way of an aperture 161a at an outer portion of the applicator capsule 100 and an aperture 161b at an inner portion of the applicator capsule 100. In the shown embodiment, the interior 162 of the applicator capsule 100 occupies a substantial or majority of the volume of the applicator capsule 100, however, in other embodiments, the interior 162 can occupy a lesser or minority of the volume of the applicator capsule 100 without departing from the scope and spirit of the disclosure. The insert 140 can include an interface, such as an adapter 163, that interfaces with a correspondingly shaped interface within the interior of the body of the system to form a fluidic seal to prevent leakage of a composition therefrom. During use of the shown example system, the composition flows from the interior of the body of the system to the interior 162 of the applicator capsule 100, and at least partially fills the interior 162 until it reaches the roller ball aperture 111. The composition flows through the roller ball aperture 111 and out of the applicator capsule 100, where it contacts the roller ball 110 and the portion of skin of the subject during application.

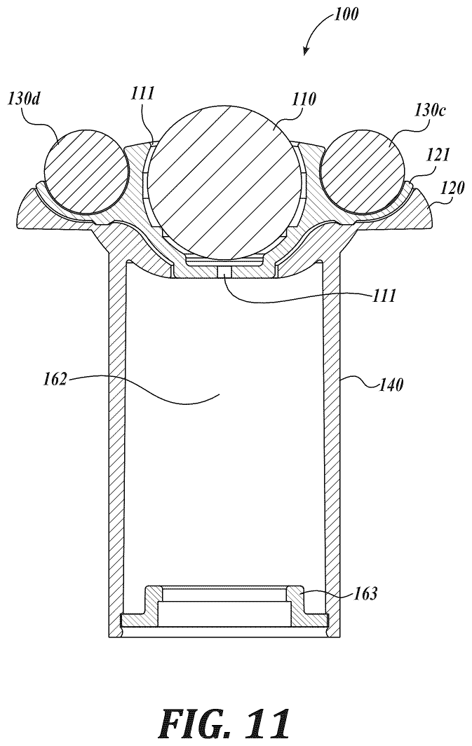

In the embodiment of FIG. 11, an interior 162 of the applicator capsule 100 is not fluidly connected to an interior of the body of the system, and does not include apertures as shown at FIG. 10 (i.e., apertures 161a, 161b). Instead, in the embodiment of FIG. 11, the interior 162 can comprise a composition therein and the adapter 163 can be coupled, on a lower portion thereof, to a piston. With an upward movement of the piston, the adapter 163 can slide upward within the interior 162 and compress the composition therein, such that the composition is expelled through roller ball aperture 11 and out of the applicator capsule 100, where it contacts the roller ball 110 and the portion of skin of the subject during application. In embodiments, after use, the applicator capsule 100 can be removed and cleaned for reuse.

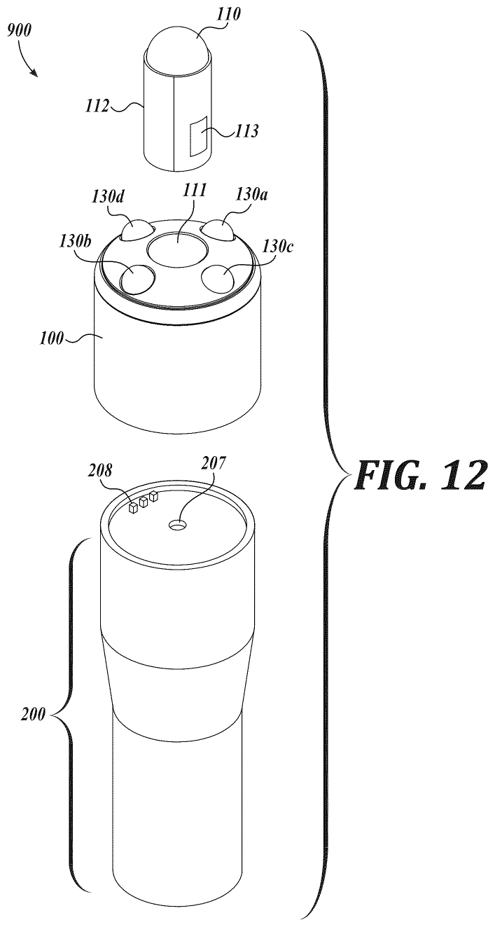

As shown at FIG. 12, an example system 900 for application of a composition to a portion of skin of a subject comprises an applicator capsule 100, comprising a plurality of electrodes (130a, 130b, 130c, 130d) configured for a transmission of an electrical current through the portion of skin of the subject, and a body 200, configured for reversible attachment of the applicator capsule 100 thereto, and a formula pod 112 reversibly that comprises a rollerball 110 configured for a rolling contact with the portion of skin of the subject. The formula pod 112 is attachable to the applicator capsule 100 and is configured to hold and release the composition, from a reservoir within the formula pod 112, based on at least one operation of a contact-less piston of the body 200 for delivery of the composition from the reservoir of the formula pod 112 to the portion of skin.

As such, the roller ball 110 can be an element of a formula pod 112, as shown by way of non-limiting example at FIG. 12. An example formula pod 112 of system 900, according to embodiments, comprises a composition for application to the skin of the subject therein, and can be inserted into an applicator capsule 100 and thereby operably connected to a body 200 of the system 900. In the shown embodiment, a piston can extend through an aperture 207 of the body 200 and an interior of the applicator capsule 100, and can mechanically expel the formula from the formula pod 112 with a movement of the piston. A power source of the body 200 can be operably connected to the applicator capsule 100 by way of an electrical connection 208, which can be configured to electrically connect with a corresponding electrical connection of the applicator capsule 100. In the shown embodiment, a formula pod microchip 113 is included and configured to enable identification of the composition in the formula pod, and/or one or more compounds thereof, for selection of a program for electroporation and/or iontophoresis application of the composition, as described herein. Since the formula pod 112 is easily removable from the device, it can be exchanged for alternative formula pods 112, e.g., alternate compositions. In the shown embodiment, the body 200 of the device may not include a reservoir as in other implementations, since the composition can be supplied by the formula pod 112, as shown.

An example application system for application of a composition to a portion of skin of a subject can include a form factor that includes a body with a body surface that is shaped and/or configured, e.g., ergonomically shaped, to be gripped by a user for operation of the system, and/or can be compact and configured to snap into or connect with attachment sites of the mask. In embodiments, an applicator capsule is attachable to the body by way of an insert attachment. The insert attachment can include a snap-on attachment, for example, such that the adapter can be inserted into the body and secured in place by the snap-on attachment of the insert attachment, or alternatively, by way of locking grooves being locked in place. Other attachment mechanisms can be implemented for the insert attachment without departing from the scope and spirit of the disclosure.

In embodiments, body 200 comprises a power source (e.g., battery or rechargeable battery) operably connected to an actuator by way of one or more electrical couplings. The actuator can mechanically actuate a piston of the system to transmit a portion of a composition of a reservoir of the system, from the reservoir through the body and to the applicator capsule. The power source can be electrically coupled to the electrodes (130a, 130b, 130c, 130d) for transmission of electric current to the electrodes based on a program of the system, for example.

In embodiments, the system can include a reservoir attachable to the body by a reservoir attachment. The reservoir is reversibly attachable to the body and can be attached, such as by way of a snap-on attachment or other attachment mechanism, and removed by way of reversal of the snap-on attachment or other attachment mechanism. The reservoir is configured to hold the composition therein, and release the composition therefrom, such that the composition flows through an interior of the body to an interior of the applicator capsule based on at least one operation of a piston or contact-less piston of the body. With movement of the composition through the interior of the body and the interior of the applicator capsule, and out the roller ball aperture, the composition can effectively flow from the reservoir through the body of the system and out the applicator capsule, where it contacts the skin and is ready to be rollingly applied to the skin and electrically transmitted to and/or through one or more layers of the skin, as described herein.

In embodiments, the system can be controlled at least in part by a user through control circuitry, which can include a power button and/or an eject button operably connected to the control circuitry by way of one or more electronic connections. In the shown embodiment, the power button can be activated by a user to power on the system or to activate a functionality of the system, such as an operation of the contact-less piston for expulsion of the composition, or a cleaning solution or liquid, from the system. In embodiments, the eject button can be activated by a user to eject the reservoir from the body of the system, for example, for replacement of the reservoir and/or cleaning or maintenance of the system or a component thereof. In embodiments, a status indicator is included and is operably connected to the control circuitry, or other circuitry of the system, for indicating a status of the system to the user. The status indicator can indicate status of one or more components of the system, such as charge level of a rechargeable battery of the system, fill level of the reservoir, need for cleaning of the system or a component thereof, and the like.

According to example embodiments a system includes the body and can include, in various aspects, an applicator capsule 100, a rollerball configured for a rolling contact with the portion of skin of the subject, and a plurality of electrodes. The system can include one or more electrodes configured or configurable for a radiofrequency (RF) warming or ultrasound electrical transmission, as well as one or more electrodes configured or configurable for an electroporesis and/or iontophoresis electrical transmission, as in the shown embodiment. The electrodes can be operably connected to control circuitry of the system, as well as a power source of the system, such as a rechargeable battery, for the generation of one or more current patterns. The one or more current patterns can be at least a part of a program for delivery of one or more molecules of the composition of the reservoir to the portion of skin of the subject. The program can be selected, by the control circuitry, based at least in part on the composition (or an identifying characteristic thereof), as described herein.

In embodiments, a formula pod can include a microchip that corresponds to one or more compounds of a composition within the formula pod. The system can select, based on an identifying characteristic of the composition obtainable by the system from the microchip, a program for administration of therapeutic electrical current(s). Characteristics of the composition can include one or more molecular weights of one or more compounds, one or more ionic states of one or more compounds at a given pH and ionic strength of the composition, or the like. For example, larger molecules of a composition may require a stronger current to be applied to effectively carry the molecules to and through one or more layers of the skin. In embodiments, a user can control one or more modes of operation, or programs, of the system to increase or decrease current based on user preference.

In embodiments, one or more compounds of the composition can be electrically resistive to provide a degree of electrical resistance to the composition as a whole; in this manner, electrical current of an RF warming current or ultrasound electrical transmission encounters resistance with those one or more compounds, generating heat within the composition, which in turn warms the skin. In embodiments, the system can create an alternating electric field with an oscillating frequency, and the resulting current can be introduced to the skin via one or more electrodes. In embodiments, the current can warm the skin directly due to electrical resistance within the skin. In embodiments, the current can warm the skin indirectly, with warming of one or more compounds of the composition, and directly, in combination, for improved skin warming. In embodiments, a frequency range of 300 kilohertz (KHz) to 1 megahertz (MHz) can be used. In example implementations, a frequency of 450 KHz can be used.

Accordingly, in aspects, the disclosure provides a “smart” system comprising circuitry configured to perform all or part of a method, including but not necessarily limited to control of expulsion of the composition from the system, monitoring of a level of the composition within the reservoir, monitoring of a level of charge of the power source, and the like. In embodiments, circuitry of a device is configurable with a processor and processor-executable instructions stored on a non-transitory machine-readable medium of the device. In embodiments, a device includes a software application configured to perform all or part of one or more methods or processes of the disclosure, in any order or combination. However, in embodiments, a device includes dedicated hardware circuitry. Further configuration of circuitry of the device can include wireless communication or networking circuitry, for example, circuitry configured for a wireless connection, such as a Bluetooth® connection, a Bluetooth® low energy (BLE) connection, and/or a Wi-Fi® connection, and/or a wired connection. The networking circuitry, in combination with other circuitry of the computational device, can be used to request, retrieve, and/or receive data from a computational device or a remote server, for example. In embodiments, the device can be operated with use of a computational device, such as a smartphone or personal computational device, that can be operated by a user via a graphical user interface, as known in the art. In embodiments, the circuitry can include operable connection of one or more sensors with the processor, or other circuitry, for performing logic operations and/or methods based on data received from the one or more sensors, for example.

An example system includes an applicator capsule operably connectable to the body, by way of one or more operable connections, e.g., physical, fluid, and electronic connections, for electronic and fluid communication between the capsule and the body of the device. The applicator capsule includes one or more electrodes thereon that are operably connected to control circuitry of the system for delivery of a microcurrent, e.g., for transmission of electroporation current, iontophoresis current, RF warming current, or any combination thereof, to the portion of skin of the subject.

In embodiments, the system includes a computer or computational circuitry, which can comprise, control, and/or direct control circuitry, or other circuitry of the system, for carrying out one or more operations of the system. Computational circuitry can include hardware circuitry, processor circuitry, or both, configured for execution of logic operations, such as activation and deactivation of an electromagnet for movement of the contact-less piston and expulsion of the composition from the system, selection and execution of one or more programs for delivery of microcurrent (e.g., electroporation, iontophoresis, and/or RF warming current), monitoring charge status of a power source, monitoring percentage of the composition utilized in the reservoir, and the like. Computational circuitry can comprise a non-transitory computer-readable medium having stored thereon instructions which, when executed by one or more processors, configure one or more processors for performance of all or part of a method or operation of the disclosure, in whole or in part, with the steps being in any order. The system can be configured to accept user input from a user, which can include an activation or deactivation signal (e.g., with use of a power button), a status inquiry signal (e.g., with use of a power button or other control element), or the like.

Computational circuitry controls, coordinates, directs, or otherwise enables transmission of microcurrents as part of execution of programs for administration of compositions to the skin. In embodiments, computational circuitry enables transmission of one or more pulses for delivery of current that is high voltage and/or high frequency, by way of electroporation circuitry and/or microcurrent circuitry. The computational circuitry also controls, coordinates, directs, or otherwise enables transmission of RF warming current by way of ultrasound transducer circuitry. An electrode can be configured and used for electroporesis, iontophoresis, or both (sequentially and/or simultaneously), and the ultrasound transducer circuitry can be configured and used for RF warming current transmission.

In embodiments, computational circuitry can be configured to electronically control at least one operation of the contact-less piston. For example, computational circuitry can, as part of execution of at least one program for application of a composition, deliver an electric current to an electromagnet of the system to activate the electromagnet and attract or repulse the contact-less piston, which can be magnetic and/or operably connected to a magnet, for movement of the contact-less piston and displacement of the composition in the body of the system.

In embodiments, computational circuitry can be operably connected to and configured to receive a signal from a microchip of the reservoir that corresponds with one or more compounds of the composition in the reservoir. The control circuitry can thereby identify what one or more compounds are in the composition that are to be delivered to and/or through the skin, and can electronically control the transmission of the electrical current, based on a program selected by the control circuitry, for delivery of the one or more compounds to the portion of skin of the subject. For example, as part of an example program selection of an example system, a capsule can be attached to the body of the device for delivery of a microcurrent based on an identification of the composition or a compound thereof. Control circuitry of the device can receive a signal from the microchip, through a wired and/or wireless transmission, for example, a radiofrequency identification (RFID) process, that corresponds to identity of one or more compounds of the composition, or the composition as a whole. The system can select a program for delivery of microcurrent based on the identity of the one or more compounds or the composition as a whole. For example, if one or more large molecules are present in the composition as active ingredients to be delivered into the skin, the system can select a program comprising electroporation of the large molecules. If one or more small molecules are present in the composition as active ingredients to be delivered into the skin, the system can select a program comprising microcurrent, e.g., iontophoresis. In embodiments, a program that includes only electroporation (and not microcurrent) is selected. In embodiments, a program that includes only microcurrent (and not electroporation) is selected. In embodiments, a program that includes electroporation and microcurrent is selected. In this manner, in embodiments, one or more compounds of the composition comprises a large molecule, a small molecule, or both, and the program configures the control circuitry for transmission of electrical current based on the signal, e.g., received from the microchip of the reservoir.

Accordingly, in embodiments, the iontophoresis delivers a large molecule of the composition, a small molecule of the composition, or both, through a dermal layer of the portion of skin. In embodiments, the electrical current comprises a radiofrequency (RF) warming electrical current. In embodiments, the RF warming electrical current creates one or more microchannels in a dermal layer of the portion of skin. In embodiments, the RF warming electrical current is transmitted at a frequency in the range of 100 kHz to 1 MHz.

In another aspect, the disclosure provides a method for administering a composition to a portion of skin of a subject, the method comprising: receiving, with control circuitry of a system, a signal from a microchip of a reservoir of the system, wherein the reservoir contains at least a portion of the composition therein and the signal corresponds with one or more compounds of the composition in the reservoir; activating, with control circuitry of the system, an electromagnet of the system to move a contact-less piston of the system and displace at least the portion of the composition from the reservoir through an interior of a body of the system to an interior of an applicator capsule of the system for expulsion of at least the portion of the composition from one or more apertures of the applicator capsule; and transmitting, with control circuitry of the system, an electrical current through the portion of skin of the subject, wherein transmission of the electrical current is based on a program selected by the control circuitry based at least in part on the signal. According to various examples, a method of using an application system of the disclosure to apply a composition to a portion of a subject's skin is contemplated. An applicator or reservoir filled with formula or composition is attached to the dispensing device comprising the body and other elements of the system. The formula is identified by control circuitry of the system as it reads a contactless chip by way of receipt of the signal, for example, with a contactless reader (e.g., RFID reader). Generation of a magnetic field is used to move the piston and displace the composition from the reservoir. The composition is dispensed from the system as the piston moves. One or more microcurrent treatments are implemented, and a formula can be applied with or without a roller ball.

In embodiments, an electrical impedance can be delivered as at least part of an RF warming microcurrent, which can range from about 10-50 kOhm. In embodiments, electrical pulses can be delivered in short bursts, for example, 1 msec burst for every 100 msec of time (1% load). In embodiments, a 1% load, a 2% load, a 3% load, a 4% load, a 5% load, a 6% load, a 7% load, an 8% load, a 9% load, 10% load, an 11% load, a 12% load, a 13% load, a 14% load, a 15% load, a 16% load, a 17% load, an 18% load, a 19% load, a 20% load, a 21% load, a 22% load, a 23% load, a 24% load, a 25% load, or a higher percentage load, can be applied for an RF warming microcurrent. Percentage load can be calculated by dividing the electrical burst time by the total time and multiplying the result by 100%. As would be understood by a person having ordinary skill in the art, a higher intensity RF warming microcurrent can be applied at a lower percentage load to avoid overheating the skin; similarly, a lower intensity RF warming microcurrent can be applied at a higher percentage load to ensure effective heating of the skin.

In embodiments, electroporation is delivered before, during, and/or after iontophoresis and/or RF warming. In embodiments, iontophoresis is delivered before, during, and/or after electroporation and/or RF warming. In embodiments, RF warming is delivered before, during, and/or after electroporation and/or iontophoresis.

As an example method, one or more electrode pairs of the system can be operated in a concurrent mode, as follows:
RF warming ON for 90% load;
RF warming OFF, during which electroporesis and/or iontophoresis is applied.

As another example method, one or more electrode pairs of the system can be operated in a sequential mode, as follows:
RF warming ON for 50% load;
RF warming OFF, during which the skin cools enough to retain warming effect and pore opening;
Electroporesis and/or iontophoresis is applied.

An example applicator capsule can include an example piston assembly. A capsule can include a cap, a rollerball, a fitment, a body, a piston, a plug, a push rod, and an authentication chip. The shown example piston assembly, and other structures shown for enabling movement of the composition within the capsule, can be incorporated into various embodiments that include a rollerball and electrodes, as described herein. In embodiments, the cap can be disposed at a first end of the body and configured to attach to the body. For example, the cap can be threaded and twist tightened onto the body which can also be threaded (as shown) or the cap can be snap tightened onto the body. The fitment can be disposed at the first end of the body. The cap, fitment, body, piston, and plug can be fabricated from a polymer material. Non-limiting examples of materials for the cap, fitment, body, piston, and plug (either separately or together) can include a thermoplastic elastomer, polypropylene (PP), polyethylene terephthalate (PETG), acrylonitrile butadiene styrene (ABS), polycarbonate (PC), polyamide (Nylon), polystyrene (PS), low-density polyethylene (LDPE), high-density polyethylene (HDPE), or any combination thereof. For example, all pieces can be fabricated from PP. In another example, the cap can be fabricated from PP, the fitment can be fabricated from PETG, the body can be fabricated from PP, the piston can be fabricated from LDPE, and the plug can be fabricated from PP.

The body can be shaped substantially cylindrical and can include a first opening at the first end and a reservoir at a second end, wherein an inner diameter of the first opening is wider than an inner diameter of the reservoir. Both the first opening and reservoir can be substantially annular. The first opening of the body can include a length of substantially straight stroke having the inner diameter of the first opening. The first opening can taper more narrowly down to the inner diameter of the reservoir. The reservoir can be substantially straight and connected to the tapered portion extending from the first length of substantially straight stroke. It can be appreciated that the cross-sectional shape of the body can be fabricated as any of a myriad of other shapes, for example triangular, square, pentagonal, hexagonal, octagonal, or the like.

The first opening can be configured to hold the fitment. The fitment can include an exterior shape that is configured to be push-fit into the first opening, wherein the fitment shape can contour to the length of substantially straight stroke and the tapered portion extending from the length of substantially straight stroke. Thus, the fitment can form a liquid-tight seal with the first end of the body. In embodiments, the fitment can be fabricated as part of the body at the first end of the body. For example, the fitment and body can be molded together as one piece. The fitment can be configured to hold the rollerball, wherein an interior shape of the fitment is substantially hemispherical. A first end of the fitment can include a rollerball retainer. The rollerball retainer can be an annular extrusion of material from the first end of the fitment that can slightly taper inwards towards the interior of the fitment such that the inner diameter of the rollerball retainer is narrower than the diameter of the rollerball. The rollerball can be installed in the fitment by pushing the rollerball through the opening of the rollerball retainer. The rollerball retainer can elastically deform outwards (i.e., the rollerball retainer opening widens and may thus be fabricated from a deformable polymer) to accommodate the rollerball when the rollerball is pushed through and then return to its original inner diameter. The rollerball can be fabricated from glass, metal, or a polymer, such as the ones described for the cap, fitment, body, piston, and plug.

The piston and plug can be disposed at the second end of the body. The piston can be shaped substantially disc-like and can include an outer diameter equal to, or marginally narrower than, the inner diameter of the reservoir such that a liquid-tight seal can be formed between the piston and an interior of the reservoir. The plug can also be shaped substantially disc-like. The piston can be installed in the reservoir and the plug can be installed at the second end of the body, wherein the plug prevents egress of the piston. The plug can be push-fit, snap-fit, twist-tightened, or chemically attached to the second end of the body. The plug can include a hole in the middle configured to allow the push rod to reversibly travel through. In another aspect, the plug can be fabricated as a part of the body at the second end of the body. For example, the plug and body can be molded together as one piece. It can be appreciated that the piston and plug can be shaped according to the cross-sectional shape of the body, and the disc-like shape is just one example.

A first end of the push rod can be configured to abut the piston. For example, the piston can include a molded indentation opening towards the second end of the body having a shape complementary to the first end of the push rod. A second end of the push rod (not shown) can be attached to a metering device (not shown) configured to translate the push rod a predetermined distance. The metering device can take the form of an applicator, and such an applicator would be configured to receive the capsule. The abutting of the first end of the push rod against the piston therefore causes the piston to travel towards the first end of the body (i.e., into the reservoir) the same predetermined distance the push rod is translated.

The reservoir can be configured to hold a composition, e.g., a solution. In embodiments, the solution can be a cosmetic. In embodiments, the composition can comprise one or more small molecules to be delivered to and/or through the skin, one or more large molecules to be delivered to and/or through the skin, or any combination thereof.

In embodiments, the composition can be a topical medication, such as a serum, an ointment, a lotion, oil, an essential oil, a cream, a gel, a paste, foam, a water-based mixture, and an alcohol-based mixture (e.g., a tincture), or any combination thereof. The topical medication can include active ingredients, such as drug content, for treating skin ailments, and/or can include nutrients, for example, vitamins and minerals.

A capsule can include a fitment which can include a well. The well can be the volume between the rollerball and the interior of the fitment. The well can be configured to receive a predetermined volume of solution from the reservoir. The fitment can include a solution regulator disposed at a second end of the fitment through which the predetermined volume of solution is transferred from the reservoir to the well. The solution regulator can be an orifice or a partially open orifice through which the solution flows towards the well, wherein the solution regulator can be configured to meter the predetermined volume of solution passing through and preventing undesired reverse flow of solution from the well towards the reservoir.

In embodiments, the solution regulator can be provided by a check valve. The solution regulator can be substantially open and configured to allow attachment or insertion of the check valve. The check valve can be installed inside or proximal to the second end of the fitment and held in place via a check valve holder. The check valve and check valve holder can be installed in the reservoir through the second end of the body. For example, the check valve can be installed first and the check valve holder can be installed after, wherein the check valve holder includes features that allow it to be snap fit into complementary features of the reservoir. In another non-limiting example, the check valve can be coupled to the check valve holder prior to installation of both into the reservoir. In another non-limiting example, the check valve and check valve holder can be chemically bonded to the reservoir by, for example, glue, epoxy, caulking, or any combination thereof. In embodiments, the check valve and check valve holder can be fabricated as a single part, i.e., the check valve includes features that allow it to be snap fit into the complementary features of the reservoir without requiring the separate check valve holder. Non-limiting examples of materials for the check valve holder include a thermoplastic elastomer, PP, PETG, ABS, PC, Nylon, PS, LDPE, HDPE, and any combination thereof.

In embodiments, the check valve can be a one-way valve allowing solution transfer in a single direction of flow (or preventing solution transfer in said direction of flow when flow stoppage is desired). The check valve can be a deformable membrane held in position via tension, wherein the position in tension forms a liquid-tight seal. For example, the check valve can be fabricated from LDPE or PETG. In response to a force applied on the deformable membrane originating from a single direction, the membrane can deflect along the direction of the applied force. Upon release/ceasing of the applied force, the tension on the membrane can return the membrane to its un-deflected orientation. Thus, the check valve can be in one of two states. A first state can be closed and liquid-tight, wherein the check valve does not allow solution from the reservoir to transfer to the well. It can be appreciated by those in the art that other one-way valves can be used without departing from the scope and spirit of the disclosure, for example, a spring-ball construction. A second state of the check valve can be open, wherein the check valve membrane is deflected, thereby breaking the liquid-tight seal and allowing solution to transfer through the check valve.

In embodiments, the push rod can be translated a predetermined distance. The push rod can concomitantly translate the piston the predetermined distance in the direction of the first end of the body. Since the solution in the reservoir may not be compressible, the force of the piston pushing on the solution can result in the check valve switching from the first (closed) state to the second (open) state. The open check valve can then allow the predetermined volume of solution to transfer from the reservoir to the well. The rollerball can be spherical and include a first portion of surface area in contact with the solution that was transferred to the well. The rollerball can include a second portion of surface area exposed to the exterior and configured to contact a user's skin. The rollerball can be configured to roll across the user's skin and transfer the predetermined volume of solution, for example the topical medication, from the well to the user's skin. As the rollerball is rotated over the user's skin and deposits the solution onto the user's skin, the second portion of surface area rolls into the well and is coated again with more solution. Notably, the fitment can be fabricated to include some play between the interior of the fitment and the rollerball to allow case of rolling of the rollerball and facilitate re-coating of the rollerball without the interior of the fitment scraping off said coating of solution as the rollerball rolls.

The predetermined distance the push rod is translated can be determined by calculating the distance needed for the piston to travel in order to displace the predetermined volume of solution in the reservoir. The maximum predetermined volume of solution transferred from the reservoir can be determined by calculating the volume of solution the well is capable of holding. The predetermined volume of solution actually transferred from the reservoir to the well can be determined by the metering device, for example the user can be attempting to complete a recommended regimen for treating a skin ailment. Thus, the user may desire a specific dosage of topical medication for applying to the user's skin and the metering device can be configured to transfer the predetermined volume of solution from the reservoir to the well at a predetermined frequency. For example, the metering device can transfer 0.3 mL of solution on a daily basis during a 14-day treatment plan, wherein the metering device is configured to allow the user to apply the solution within a preset length of time, for example 3 minutes per day. An on-board chip in the metering device can record the user's usage and a position of the piston, wherein upon determining that the position of the piston correlates to a 14th day of the treatment, the metering device may notify the user to replace the capsule. In response to determining that the user has removed the capsule, the metering device can adjust and reset the position of the piston to a position correlating to a start of the 14-day treatment plan. In addition, the metering device can reset the on-board chip to begin recording the user's usage again anew.

Advantageously, the built-in solution regulating feature, i.e., the check valve, can prevent excess solution from transferring to the well once the piston stops and the release of force (and the tension on the check valve) closes the check valve. Therefore, this prevents the user from over-applying the solution, which can be important when the solution is a topical medication including a particular active ingredient, for example a drug, which should not be dosed in excess. Additionally, this can be aided by the metering device in which the capsule is installed, wherein the metering device prevents the user from overdosing by only translating the piston via the push rod a predetermined number of instances within a predetermined timeframe, for example once per day, and not more frequently than programmed regardless of user input (e.g., the user prompting the metering device for another dose).

In embodiments, a therapy device is configured for a cold plasma treatment, for example, as described in WO 2020028329 A1, the contents of which are incorporated by reference herein in their entirety, for all purposes. Accordingly, a system and method for cosmetic treatment of a region of a biological surface using cold atmospheric plasma is presented. In embodiments, a method of treatment of a region of a biological surface with cold plasma includes: selecting a post-treatment formulation; and applying the post-treatment formulation to the region pre-treated by the cold plasma.

Portable cosmetic devices incorporating cold atmospheric plasma (additionally referred to as “cold plasma” or “plasma”), as currently available, implement limited modalities of treatment. Most focus solely on generating a plasma in proximity to a region of a person's skin as an avenue for treatment of the region. Such an approach does not leverage the therapeutic advantages and benefits of a multi-modal treatment and does not create synergistic therapy outcomes that exceed the effect of the individual treatment modalities in isolation. In embodiments of the present technology, a multi-modal cold plasma device includes one or more auxiliary or additional mechanisms (vibration, heat, etc.) in addition to generating the cold plasma.

A multi-modal plasma device that takes advantage of synergistic treatment effects will achieve enhanced results over a shortened treatment duration and a reduced power demand, relative to a mono-modal plasma device. What is more, a multi-modal device mitigates the potential risks of excessive exposure to potentially harmful species generated in the plasma, such as oxidizing species or reactive radicals, by enhancing permeability of plasma generated species, or by providing secondary treatment that does not rely on plasma generated species for efficacy.

Blemishes develop for multiple reasons, some of which include harmful bacteria, blocked pores, irritation, imbalance in moisture or oil, etc. Typically, blemishes develop from an easily treatable early stage to a painful and difficult condition within a single day, often within hours of first sensing a new blemish. Without being bound to theory, it is believed that exposure to a cold atmospheric plasma and species generated therein will effectively treat blemishes. It is further believed that rapid treatment at the site of the blemish, immediately upon detection, prevents the growth of unsightly blemishes that may leave permanent scarring.

Cold plasma devices designed to treat large regions of the skin, incorporating internal power sources sufficient for prolonged use, are too large and too heavy to conveniently carry on a regular basis. While well suited for a cosmetic routine that takes place at home, their size and weight makes them less suited for rapid response to a developing blemish or acne sore that is discovered when away from the home. It is believed that a small area plasma device designed for rapid treatment of individual blemishes, also called “spot treatments,” will effectively treat a blemish in its early stages and will be portable and convenient.

Direct exposure of a region of biological surface to cold atmospheric plasma can be supplemented with therapy regimes (also referred to as “formulations” or “therapeutic formulations”). For example, plasma generated species, such as ultraviolet photons or reactive oxygen species, may damage biological surfaces. Therefore, therapeutic formulations can be used to prevent or minimize damage to biological surfaces.

In embodiments, a pre-treatment formulation protects the region against potentially harmful plasma generated species. In another embodiment, a post-treatment formulation neutralizes plasma generated acids that may harm the biological surface following treatment. In embodiments, plasma-activated media provides effective therapy when applied to the region without direct exposure to the plasma. Without being bound to theory, it is believed that cold atmospheric plasma produces reactive species and secondary reaction products that remain in liquid media following exposure to the plasma.

While a basic plasma treatment is thought to beneficially impact biological systems, the risks of over-exposure or inadvertent damage are non-negligible. Accordingly, conventional plasma devices limit a consumer's ability to implement customized treatment. These treatment risks can be mitigated by customizing the plasma for a specific therapeutic result being sought. In embodiments, a size of the target treatment area is selectable. In other embodiments, a chemical intermediary may modify the concentrations of potentially harmful species generated in the plasma. In practice, surface conditions and plasma parameters are coupled, where variation in one induces changes in the other. A sudden shift in surface moisture, for example, may affect electrical conductivity of the surface and lead to an increase in plasma intensity. Conversely, a sudden increase in plasma intensity may vaporize moisture from the surface, in turn changing the properties of plasma. This variability and multi-parameter coupling necessitates control of the plasma treatment device.

Complex interactions between light emission from the plasma, plasma generated species, and biological chemicals native to biological surfaces further complicates cold plasma therapy. In some cases, plasma generated species may acidify a biological surface, thereby aggravating preexisting conditions and outweighing any beneficial outcomes of plasma treatment, for example by light emission, or by exposure to plasma generated species that stimulate wound healing or that would otherwise denature harmful bacteria present in the biological surface. However, measurement of the plasma treatment on a biological surface may allow modulation of the plasma, therefore making the treatment more effective and safer.

Uniformity and steady application of cold plasma during treatment is desirable for several reasons. Non-limiting examples include providing a predictable dose of plasma generated species to the biological surface, providing a consistent treatment result despite variability in conditions of the biological surface, maintaining a uniform exposure to the cold plasma across a region of a biological surface, etc.

### Cold Plasma Therapy Devices

Non-thermal “cold” atmospheric plasma can interact with living tissue and cells during therapeutic treatment in multiple ways. Among the possible applications, cold atmospheric plasma can be used in biology and medicine for sterilization, disinfection, decontamination, and plasma-mediated wound healing.

Several commercialized devices are certified for medical treatment at the present time. These devices are not designed for home use by consumers. Instead, they are designed for use by medical technicians with expertise and training in medical treatment techniques. An example of such device is Rhytec Portrait®, which is a plasma jet tool for topical dermatological treatments. This device features complex power supplies with tightly regulated parameters, using radio-frequency power sources. In addition, the Bovie J-Plasma®, the Canady Helios Cold Plasma, and the Hybrid Plasma™ Scalpel are all available for use as medical treatment devices. In Germany, the kINPen®, also a plasma jet device, and the PlasmaDerm®, a dielectric barrier discharge (DBD) device, are both certified medical devices that have been introduced to the market within recent years. These devices aim at medical treatment of human tissues, either externally, as in the PlasmaDerm®, or internally. In contrast with the plasma devices for the medical use, the devices for the cosmetic use are geared for a generally intuitive use by consumers, resulting in cosmetic care and pleasant sensation, as opposed to well controlled and certifiable therapeutic effect.

A plasma generator can be implemented in accordance with prior art. A cold plasma forms through disparate excitation of electrons in a plasma gas by electric fields, relative to the milder excitation effect of the fields on the more massive nuclei of the plasma gas. The cold plasma is formed between a live electrode and a ground electrode, also called a counter-electrode, when the live electrode is energized relative to the ground electrode by a power source. The power source is an alternating current source or an amplitude modulated direct current source. The cold plasma is a dielectric barrier discharge if the plasma generator includes a dielectric barrier that is placed against the live electrode. The cold plasma contains both high temperature electrons and low temperature ions and neutral species. In conventional systems, the plasma gas includes noble gases like helium or argon, and also oxygen and nitrogen containing gases to form reactive oxygen and nitrogen species (RONS). In some cases, as with the PlasmaDerm®, the plasma forms directly in air.

A dielectric barrier discharges in operation. The plasma forms as multiple discrete filamentary discharges that individually form conductive bridges for ions and electrons to migrate between the electrodes.

For topical treatment, several forms of plasma are used. The first is the gas jet plasma which provides a jet of ions and reactive species that can be directed to a target over varying distances, typically at distances greater than a few millimeters. The medical plasmas described in a preceding paragraph typically feature a gas jet plasma. A second form is the Floating Electrode Dielectric Barrier Discharge (FE-DBD) devices, in which the target substrate (often the human body) acts as a floating ground electrode. The third form is a DBD plasma wand, where the dielectric barrier is placed against a floating ground, instead of the live electrode, and may take the form of a fluorescent tube. The fourth form is a coordinated plurality of dielectric barrier discharge sources. In such an arrangement, a number of atmospheric FE-DBD plasma sources are incorporated into a handheld or flexible device, that is then used to treat one or more anatomical regions.

A cold plasma system can be implemented. A skin treatment device produces cold plasma through a unitary structure that includes a head and a body. The device includes one or more user controls, including a plasma power switch, and a light switch. The head includes one or more light emitting diodes (LEDs). The skin treatment device further includes a plasma pulse control, configured to create the plasma at the head while the plasma pulse control is pressed. The skin treatment device includes a charging port for charging an enclosed battery. The skin treatment device includes internal electronic components that drive the plasma.

Electronic components can include a unitary structure having a DBD head and body. The cold plasma is produced between electrodes included in the DBD head, which serves as the treatment site. The DBD head is electrically connected to a high voltage unit, providing power to the DBD head. The power needed to drive the plasma is provided by a rechargeable battery pack enclosed within the body. The system includes one or more LEDs, connected to the system through a main PC board and control circuitry. The main PC board and control circuitry controls the flow of electricity to the LED and the high voltage unit, and receives input from one or more user controls and external power in to charge the rechargeable battery pack.

Without being bound to theory, it is believed that the effect of cold atmospheric plasma therapy is due to some extent to interaction between RONS and biological systems. A non-exhaustive list of RONS includes: hydroxyl (OH), atomic oxygen (O), singlet delta oxygen (02(¹D)), superoxide (O2), hydrogen peroxide (H2O2), and nitric oxide (NO). Hydroxyl radical attack is believed to result in peroxidation of cell membrane lipids, in turn affecting cell-cell interaction, regulation of membrane-protein expression, and many other cellular processes. Hydrogen peroxide is a strong oxidizer, believed to have a harmful effect on biological systems. Nitric oxide is believed to play a role in cell-cell signaling and bio-regulation. At the cellular level, nitric oxide is believed to affect regulation of immune deficiencies, cell proliferation, phagocytosis, collagen synthesis, and angiogenesis. At the system level, nitric oxide is a potent vasodilator.

Cold atmospheric plasmas also expose biological surfaces to electric fields, on the order of 1-10 kV/cm. It is believed that cells respond to such fields by opening trans-membrane pores. Such electric-field induced cellular electroporation is believed to play a role in transfusion of molecules across cell membranes. Without being bound to theory, the efficacy of treatment is believed to be due at least in part to long-lived plasma-generated species, which in an air plasma will be a variety of RONS at concentrations particular to the operating parameters of the cold atmospheric plasma source.

While cold atmospheric plasma can also be used to ablate tissue or effect treatment in a very short time when operated at high power and intensity, such treatment is believed to harm surrounding tissue and to penetrate far beyond the treated area. Without being bound to theory, it is believed that cold atmospheric plasma treatment at low intensity avoids damaging cells.

Without being bound to theory, it is believed that an important parameter both for direct cold atmospheric plasma treatment and for indirect treatment using plasma-treated media is the dose of plasma species imparted to the treatment surface. In general, this is expressed as a concentration of a given plasma species produced by the cold atmospheric plasma source that is imparted to a unit area of the treated surface over a unit time.

Alternatively, the dose can be expressed as a simple length of time, if the treatment has been determined and the behavior of the cold atmospheric plasma source is well understood. For example, for a stable cold atmospheric plasma source and a uniform surface, a particular dose of a given RONS will be achieved after the cold atmospheric plasma has treated the uniform surface for a given length of time. In practice, surface conditions and plasma characteristics are coupled, where variation in one induces changes in the other. A sudden shift in surface moisture, for example, may affect the conductivity of the surface and lead to an increase in plasma intensity. Conversely, a sudden increase in plasma intensity may vaporize moisture from the surface, producing RONS and changes in the surface. This variability necessitates control of the plasma treatment device, as discussed in greater detail below.

Without being bound to theory, it is believed that cold atmospheric plasma treatment penetrates into the treatment surface through a synergistic effect of electroporation, permeability of plasma generated species, and cell-to-cell signaling. The so called “bystander effect” is thought to play a role in propagating plasma induced cellular changes away from the treatment surface and into a volume beneath it. The bystander effect is believed to occur through chemical signals passed between cells in response to the introduction of a biologically active chemical, potentially amplifying the magnitude of the treatment impact.

In experiments it has been shown that RONS include reactive nitrogen species (RNS) and reactive oxygen species (ROS) that are believed to interact in differing ways to diverse biological surfaces. In agarose films, for example, RONS permeate a volume beneath the film, while in living tissues, only RNS will do so. ROS do penetrate, however, into gelatin and other liquids. ROS, being more reactive than RNS are shorter-lived and are believed to be linked in some circumstances to aggressive or harmful effects on biological surfaces, as previously discussed with respect to hydrogen peroxide.

Cold Plasma Treatment with Additional Treatment Devices

In embodiments, a cold plasma system for treating a region of a biological surface includes a plasma treatment device, including an electrode and a dielectric barrier having a first side that faces the electrode and a second side that faces away from the electrode. The system may include an auxiliary treatment device configured to enhance effects of the cold plasma on the region. In an aspect, the auxiliary treatment device is selected from a group consisting of a vibration device, a light source configured to illuminate the region, and a source of air directed to the region.

In an aspect the vibration device includes a multi-axis eccentric mass vibrator. The vibration device may include a piezo-electric actuator.

In an aspect, the light source includes one or more light-emitting diodes, configured to direct light having a wavelength in a range of 400-500 nm toward the region.

In an aspect, the source of air includes a conduit configured to direct a stream of air toward the region, an air mover disposed within the conduit, and one or more temperature control elements configured to regulate temperature of the stream of air. The air mover may include a fan. The temperature control elements may include at least one of a thermal-resistive heater coil and a Peltier cooler.

In an aspect, the plasma treatment device further includes one or more actuating members, configured to repeatedly strain and relax the biological surface in the region. In an aspect, the plasma treatment device further includes a cover placed over the dielectric barrier, and wherein the cover is configured to protect the dielectric barrier from contact damage or contamination. The cover may include glass, plastic, or quartz.

In an aspect, the biological surface includes at least one of: skin, hair, and fingernails.

In an aspect, the plasma is discharged at least partially into the biological surface.

In embodiments a method of treating a region of a biological surface with a cold plasma system includes generating the plasma by a plasma treatment device that comprises a plasma generator. In embodiments, the method includes applying the plasma to the biological surface, activating an auxiliary treatment device, treating the biological surface with the auxiliary treatment device, and turning off the plasma.

In an aspect, treating the biological surface with the auxiliary treatment device includes vibrating the plasma treatment device.

In an aspect, treating the biological surface with the auxiliary treatment device includes vibrating the biological surface at or near the region.

In an aspect, treating the biological surface with the auxiliary treatment device includes irradiating the region with light from a source of light disposed on the plasma treatment device. The source of light may emit visible light. The source of light may emit infrared light.

In an aspect, treating the biological surface with the second treatment device includes providing a stream of flowing air to the region via a conduit disposed within the plasma treatment device and an air mover disposed within the conduit. In an aspect, the method includes cooling or heating the stream of flowing air with one or more temperature control elements configured in the conduit.

Cold Plasma Treatment System with External Support Devices

In embodiments, a cold plasma system for treating a region of a biological surface includes a plasma treatment device, including an electrode and a dielectric barrier having a first side that faces the electrode and a second side that faces away from the electrode. In an aspect, the plasma treatment device further includes a rechargeable battery electrically connected to the electrode and operably coupled to the power cell via a charging link. In embodiments, the plasma treatment system further includes a support device external to the plasma treatment device. In embodiments, the support device includes a power cell electrically connected to the plasma treatment device and a controller operably coupled to the power cell and to the plasma treatment device. In an aspect, the support device is wirelessly connected to the plasma treatment device. In an aspect, the support device is electrically connected to the plasma treatment device via a detachable cable.

In an aspect, the dielectric barrier and the electrode are disposed on a retractable support, the extension of which positions the second side of the dielectric barrier for treatment of the region, the retractable support being disposed within a housing. In an aspect, the plasma treatment device is a lipstick-size device.

In an aspect, the support device is a smart device. The smart device can be selected from a group consisting of: a smart phone, a tablet, a laptop, an electronic hair-styling device, and a plasma device including a charging dock.

In an aspect, the plasma is discharged at least partially into the biological surface.

In an aspect, the biological surface includes at least one of: skin, hair, or fingernails.

In embodiments, a cold atmospheric plasma system for treating a region of a biological surface includes a plasma treatment device. The plasma treatment device may include an electrode and a dielectric barrier having a first side that faces the electrode and a second side that faces away from the electrode. In embodiments, the dielectric barrier and the electrode are disposed on a retractable support the extension of which positions the second side of the dielectric barrier for treatment of the region, the retractable support being disposed within a housing. In embodiments, the plasma treatment device includes a battery electrically connected to the electrode and a controller operably coupled to the battery and the electrode, configured to receive data and to send control inputs. The system can be a lipstick-sized system.

In some aspects, the battery is a rechargeable battery, enclosed within the device.

In some aspects, the plasma treatment device is configured to connect to a support device external to the plasma treatment device via a detachable cable. The support device can be configured to provide power and control inputs to the plasma treatment device.

In embodiments, a method of treatment of a region of a biological surface with a cold atmospheric plasma system includes attaching a detachable cable to the plasma system and transferring power and control inputs to the plasma system from a support device via the detachable cable. In embodiments, the method further includes generating a cold atmospheric plasma between the plasma system and the region, switching off the cold atmospheric plasma, and detaching the detachable cable from the plasma system.

In some aspects, the method includes extending a retractable support carrying an electrode and a dielectric barrier, the support being disposed within the plasma treatment device, that when extended places the dielectric barrier in position to generate the cold atmospheric plasma in proximity to the region. The method may include retracting the retractable support.

In some aspects, the method includes transferring power wirelessly to the plasma system, wherein the plasma system includes a rechargeable battery.

Cold Plasma Treatment System with Formulation Dispensing

In embodiments, a cold plasma system for treating a region of a biological surface, the system includes a plasma generator and a pre-treatment formulation. In embodiments, the cold plasma system includes an electrode and a dielectric barrier having a first side that faces the electrode and a second side that faces away from the electrode. In an aspect, the system further comprises a post-treatment formulation configured for applying onto the region of the biological surface.

The pre-treatment formulation can be configured for applying onto the region of the biological surface. In an aspect, the pre-treatment formulation comprises plasma treated species. In an aspect, the pre-treatment formulation comprises one or more of a fragrance, an essential oil, a pigment, and an active ingredient.

In an aspect, the biological surface includes at least one of: skin, hair, and fingernails.

In an aspect, the plasma is discharged at least partially into the pre-treatment formulation.

In embodiments, a method of treatment of a region of a biological surface with a cold plasma includes selecting a pre-treatment formulation, applying the pre-treatment formulation to the region, generating a cold plasma between a plasma treatment device and the pre-treatment formulation, and switching off the cold plasma.

In an aspect, the method includes removing the pre-treatment formulation from the region. The method may include, prior to applying the pretreatment formulation to the region, treating the pre-treatment formulation by discharging the cold plasma into the pre-treatment formulation. In an aspect, the method includes selecting a post-treatment formulation and, after switching off the cold plasma, applying the post-treatment formulation to the region.

In an aspect, the method includes removing the post-treatment formulation from the region.

In embodiments, a method of treatment of a region of a biological surface with a cold-plasma treated formulation includes selecting a formulation, applying the cold plasma to the formulation, switching off the cold plasma, and applying the formulation to the region.

In an aspect, the method includes removing the formulation.

In an aspect, the method includes, after applying the formulation to the region, discharging the cold plasma into the formulation.

In an aspect, the method includes selecting a post-treatment formulation and applying the post-treatment formulation to the region, following application of the formulation. The method may include removing the post-treatment formulation from the region.

Cold Plasma Treatment with Modular System

In embodiments, a modular cold atmospheric plasma system for treating a region of a biological surface includes a plasma generator body and a head that is removably attached to the plasma generator body. The head may have a mounting side facing the generator body and an application side configured to face the biological surface. The application side of the head may carry an electrode and a dielectric barrier having a first side that faces the electrode and a second side that faces away from the electrode.

In some aspects, the plasma is discharged at least partially into the biological surface.

In some aspects, the biological surface includes at least one of: skin, hair, or fingernails.

In some aspects, the head is tapered from a first dimension at the mounting side to a second dimension at the application side. The second dimension can be smaller than the first dimension. In some aspects, the second dimension is larger than the first dimension, and the electrode and the dielectric barrier are configured to produce a cold atmospheric plasma across an enlarged portion of the treatment region on the biological surface with respect to a size of the application side.

In some aspects, the head is a formula-application head that includes a reservoir for a liquid formula and an exuding surface connected to the reservoir via one or more conduits, wherein the exuding surface is configured to allow the liquid formula onto the region. In some aspects, the head includes a flexible skirt at the application side of the head, and wherein the flexible skirt is configured to conform to the biological surface by compressing the flexible skirt between the biological surface and the head. The flexible skirt may include a rigid spacer that restricts the compression of the flexible skirt between the application side of the head and the biological surface.

In some aspects, the head includes a plasma filter placed between the dielectric barrier and the region. The plasma filter may include at least one of a chemical filter, an ultraviolet filter configured to block the transmission of ultraviolet photons, and a charged species filter that includes a conductive surface configured to neutralize anions, cations, and free electrons. In some aspects, the chemical filter includes at least one of activated carbon, graphene, catalyst, and radical-scavenging material. The plasma filter can be configured to at least partially cover the region of the biological surface.

In some aspects, the application side of the head includes a conformable material at least partially surrounding the dielectric barrier material, wherein the conformable material is conformable to the contours of the region. The electrode and the dielectric barrier can be conformable to the contours of the region.

In some aspects, the head includes one or more air outlets on the application side of the head, configured to provide a cushion of flowing air around the dielectric barrier, and an air mover, enclosed within the air-cushion head, configured to provide the flowing air to the air outlets.

In some aspects, the electrode is pixelated into individually activated areas capable of generating the cold plasma. The system may include a controller configured to energize the activated areas of the electrode.

In embodiments, a method of treatment of a region of a biological surface with a modular cold atmospheric plasma system includes selecting a head from a plurality of removably attachable heads, attaching the head to a generator body, generating a cold plasma between the plasma system and the region, and switching off the cold plasma.

In some aspects, the method includes wherein the head is tapered from a first dimension at a mounting side of the generator body to a second dimension at an application side of the head.

In some aspects, wherein the head carries a formula, the method further includes providing the formula at the application side of the head via an exuding opening, and applying the liquid formula to the region of the biological surface. In some aspects, wherein the head is configured to filter the plasma, the method further includes generating the cold plasma through a plasma filter and applying the cold plasma onto the region of the biological surface through the filter.

In some aspects, the method includes filtering the plasma through a liquid formula applied to the region of the biological surface.

In some aspects, wherein the head is an air-cushion head, the method includes activating an air mover that is located within the head and providing a cushion of flowing air around a dielectric barrier of the head.

In some aspects, the head is a flexible head conformable to the biological surface at the region.

In some aspects, generating the cold atmospheric plasma between the plasma system and the region includes applying a flexible skirt against the region, wherein the flexible skirt surrounds a dielectric barrier of the head and containing the cold atmospheric plasma in a space between the region, the flexible skirt, and the head.

Cold Plasma Treatment with Sensors and Controlled Plasma Generation

In embodiments, a cold plasma system for treating a region of a biological surface includes a plasma treatment device, including an electrode and a dielectric barrier having a first side that faces the electrode and a second side that faces away from the electrode. The system may include one or more sensors. The sensors may measure properties of at least one of a cold plasma, the ambient environment around the system, and the biological surface. The system may include a controller operably coupled to the plasma treatment device and to at least one of the sensors. The controller may receive input from the sensors to determine control data for the plasma treatment device and send control data to the plasma treatment device.

In an aspect, the electrode is pixelated into a plurality of areas that are individually addressable by the controller.

In an aspect, the one or more sensors include a sensor placed on or near the region of the biological surface.

In an aspect, the one or more sensors include one or more motion sensors. In an aspect, the one or more sensors include a humidity sensor configured to measure a humidity of ambient air. In an aspect, the one or more sensors include a reactive oxygen sensor. In an aspect, the one or more sensors include a light sensor. In an aspect, the one or more sensors include a plasma conductivity sensor. In an aspect, the one or more sensors include a surface temperature sensor. In an aspect, the one or more sensors include a distance sensor. In an aspect, the one or more sensors include an ion concentration sensor.

In an aspect, the plasma is discharged at least partially into the biological surface.

In an aspect, the biological surface includes at least one of: skin, hair, and fingernails.

In an aspect, the cold plasma system further includes a ballast circuit connected to the electrode.

In an aspect, the cold plasma system further includes a non-transitory computer readable medium having computer executable instructions stored thereon that, in response to execution by one or more processors of a computing device, cause the computing device to perform actions including determining a uniformity of the plasma as a function of time, determining a dose of one or more plasma generated species, and modulating the plasma as a function of time to control the uniformity and the dose.

In an aspect, the cold plasma system further includes a formulation engine that, in communication with the controller, determines a target dose of plasma and a set of plasma parameters necessary for providing an application dose and a data storage system that stores data related to the application dose.

In embodiments, a method of treatment of a region of a biological surface with a cold plasma includes generating the cold plasma between a plasma source and the region and measuring one or more treatment parameters with one or more sensors. In embodiments, the method includes determining a plasma dose from the treatment parameters, modulating one or more of the treatment parameters to adjust the plasma dose, and switching off the cold plasma.

In an aspect, the method includes at least one sensor being carried by the plasma source.

In an aspect, the method includes at least one sensor being carried by the biological surface.

In an aspect, the method includes moving the plasma source while the plasma source is generating the cold plasma.

In an aspect, the method includes providing a perceptible signal when the plasma dose is outside a safe range or an effective range.

In an aspect, the method includes determining a discharge voltage as a function of time, determining a discharge current as a function of time, determining a plasma temperature as a function of time, and determining a gas temperature near the region as a function of time.

In an aspect, the method includes, prior to generating the plasma, placing at least one of the sensors onto the biological surface at or near the region, exposing the at least one sensor to the plasma, and, after generating the plasma, removing the at least one sensor.

In an aspect, the method includes automating a determination of a treatment dose by determining the treatment dose via a formulation engine that is in communication with a data storage system, determining a set of plasma parameters for achieving the treatment dose during treatment of the region, and providing the treatment dose and the set of plasma parameters to the plasma source.

In an aspect, the method includes intermittently redefining the treatment dose by re-measuring the plasma parameters during the cold plasma treatment.

In an aspect, the method includes determining an indicator of uniformity of the plasma; and modulating one or more of the plasma parameters in response to changes in the indicator.

In an aspect, the method includes measuring the plasma parameters from a group including a current provided to the discharge, a driving frequency, a voltage waveform, a peak to peak voltage, a root mean square voltage, a plasma temperature, a gas temperature, and optical emission from the plasma.

In an aspect, the method includes generating the plasma by a plurality of pixelated electrodes arranged in a matrix, and determining a discharge power for a given pixelated electrode. In an aspect the method includes modulating the discharge power for the given pixelated electrode if the plasma has localized to the given pixelated electrode.

Cold Plasma System with Additional Treatment Devices

A cold plasma treatment system provides cosmetic treatment of a region of a biological surface of a consumer. In embodiments, the system includes a cold atmospheric plasma treatment device including a plasma generator having an electrode and a dielectric barrier.

In embodiments, the plasma treatment device includes a vibration device. Without being bound to theory, it is believed that the actuation of the vibration device provides the consumer with an enhanced treatment experience, and improves treatment efficacy by mitigating plasma non-uniformity over the region. In embodiments, the vibration device may emit ultrasound vibrations. In operation, the ultrasound may enhance the transport of active plasma species into the tissue (or across diffusion barriers, in general) therefore increasing the efficacy of cold plasma treatment.

The vibration device may vibrate the treatment device, thereby affecting the distance L between the second side of the dielectric barrier and the biological surface. In embodiments, the vibration device vibrates the treatment device in multiple axes simultaneously. In other embodiments, the vibration device vibrates the treatment device along only one axis. The vibration device may vibrate the treatment device such that the plasma moves parallel to the biological surface in one or two axes. It is believed that such movement distributes the plasma across the region, thereby improving plasma uniformity. The vibration device may include one or more vibration sources, such as a piezoelectric actuator or a multi-axis eccentric mass vibrator.

In embodiments, the plasma treatment device directly actuates the biological surface by one or more actuating members. Without being bound to theory, it is believed that repeated tension and compression of the biological surface enhances the efficacy of multimodal treatment by stimulating synergistic effects with permeability of plasma generated species and consumer experience of the treatment. The actuating members can be in direct contact with the biological surface at or near the region. In embodiments, the actuating members move in opposite directions to each other, parallel to the biological surface. The actuating members may move towards each other, in turn compressing and releasing the biological surface. The actuating members may move away from each other, in turn stretching and releasing the biological surface. In embodiments, the actuating members move both towards and away from each other, thus both stretching and compressing the biological surface. In embodiments, the plasma is generated toward the region while the actuating members actuate the biological surface.

The actuation can be in the form of the ultrasound emitted by the vibration device. In different embodiments, the ultrasound can be transmitted to the target biological surface through the actuating members capable of generating ultrasound. In embodiments, the actuating members form an annular shape with the electrode and the dielectric barrier contained within the annular shape. In other embodiments, a source of ultrasound may include multiple apertures for directing cold plasma toward the biological surface. In operation, an acoustic coupling medium may couple the source of ultrasound (e.g., the actuating members) with the biological surface. Some nonexclusive examples of such coupling media are hydrogels, solid gel pads, etc. The coupling medium can be formulated to have additional ingredients and properties to enhance the experience, such as precursors and ingredients that can be activated by plasma and/or work with it, fragrance etc. In embodiments, no coupling gel is used and the source of ultrasound couples enough energy to biological surface even in the absence of coupling medium. The actuating members may actuate the surface without plasma exposure, thereby providing a tactile experience to the consumer.

In addition to treatment by the plasma, the plasma treatment device may include a light source, configured to illuminate the region with light within the area described by the characteristic dimension T. As previously described, it is believed that irradiation of the biological surface with light having a wavelength in the range of 400-500 nm provides desirable therapeutic results for cosmetic treatment of blemishes. In embodiments, the plasma treatment device includes multiple light sources. The light source may include one or more light emitting diodes, individually emitting light having a wavelength within a target range.

The light source may include an infrared light element, providing radiative heating to the biological surface. Without being bound to theory, it is believed that radiative heating of the biological surface enhances the therapeutic effect of plasma treatment by triggering a response of the biological surface to plasma generated species and by providing an enhanced experience for the consumer.

The plasma treatment device may include a cover disposed on or over the dielectric barrier. Non-exclusively, the cover may include plastic, glass, or quartz, and may block plasma generated species from reaching the biological surface. Without being bound to theory, it is believed that the plasma may emit ultraviolet photons under certain conditions. As such, it can be desirable to block the transmission of ultraviolet photons using a cover.

In embodiments, the plasma treatment device includes a source of air that directs an air stream to the region within the area described by the characteristic dimension T. The source of air may include an air mover, such as a fan or a blower, disposed within an air conduit that is shaped to provide the air stream at the surface of the region. In embodiments, one or more temperature control elements disposed within the plasma treatment device adjust the temperature of the air. Non-limiting examples of the temperature control elements include thermoelectric cooling elements including Peltier coolers, electric heating elements including resistive heating coils, etc. In embodiments, a volatile oil is disposed within the air conduit that contains a fragrance such that, when the air mover is active, the oil imparts a pleasant aroma to the region.

### Small Size Device

In embodiments, the plasma treatment device is electrically connected to an external device having a power cell and a controller. In embodiments, the plasma treatment device is electrically connected to the external device via a cable. In embodiments, the cable carries control inputs and electrical power to the plasma treatment device. In embodiments, the cable is detachable from the plasma treatment device, the external device, or both. The power cell can be a rechargeable battery including, for example a lithium ion battery. The controller can be capable of receiving data and sending control signals to the plasma treatment device.

In embodiments, the plasma treatment device includes a battery electrically connected to the electrode. The battery can be rechargeable, charged by connecting the cable to the plasma treatment device and to a power source. Some non-limiting examples of such power source are the external device, an adapter connected to a standard wall outlet providing electricity, a solar cell, etc. In embodiments, the battery charges wirelessly. In embodiments, the battery is a commercially available battery, such as a battery of one of the A-series types (“A,” “AA,” or “AAA”).

In embodiments, the external device is a smart phone. In embodiments, the external device is a laptop or a tablet, configured to be compatible with the plasma treatment device and to provide power and control inputs to the external device. In embodiments, the external device is a cosmetic tool, including but not limited to an electronic beard trimmer, a hair iron, a hair drier, an electronic epilator, etc. The external device can be a large area plasma treatment device, as described previously, further including a charging dock for electrically connecting to the plasma treatment device. In embodiments, the charging dock is configured to accept the plasma treatment device, which can be operably mounted into the large-area device for compact charging and operation as a plasma generator. In embodiments, the electrode and the dielectric barrier are disposed behind a cover. The cover can be removable. The cover may provide protection for the dielectric barrier when the plasma treatment device is not in use.

In embodiments, the electrode and the dielectric barrier are disposed on a retractable support enclosed within the plasma treatment device. The retractable support can be configured such that when retracted, the dielectric barrier and the electrode are hidden from view and the plasma treatment device cannot be activated. The retractable support may rotate through the action of a mechanism disposed at an end of the plasma treatment device opposite to the dielectric barrier, such that the dielectric barrier emerges from the opposite end of the plasma treatment device in a manner resembling a lipstick. The plasma treatment device may have a form factor similar or comparable to a retractable lipstick tube, such that it resembles the lipstick tube when inactive. In embodiments, the retractable support is a linear slide that is configured to slide the electrode and the dielectric barrier behind the shield when not in use.

In embodiments, the plasma treatment device is controlled via a user interface in the external device. In embodiments, the external device is any type of device including a battery, a general purpose computer, and computer readable memory with instructions stored thereon that, when executed by the computer implement a method of treatment of a region of a biological surface by cold atmospheric plasma.

In embodiments, the plasma treatment device includes one or more user controls including, but not limited to, a power switch, a plasma intensity selector, and a safety switch. The plasma treatment device can be switched on and switched off using a power switch disposed on the plasma treatment device, and the plasma is generated while the plasma treatment device is on. In embodiments, a safety switch prevents the plasma treatment device from turning on until the safety switch is disengaged. In embodiments, the safety switch is a fingerprint reader. In embodiments, a plasma intensity selector permits smooth and continuous modulation of the plasma intensity, in terms of a power supplied to the electrode. In embodiments, the plasma intensity selector limits the plasma treatment device to one of a number of discrete intensity settings, in terms of incremental steps in the power supplied to the electrode.

In embodiments, the plasma treatment device includes one or more light emitting diodes (not shown), providing therapeutic light to the biological surface. In embodiments, the light emitting diodes provide blue light, in the range of 400-500 nm.

Cold Plasma with Formulation Dispensing

In embodiments, the plasma treatment device, including the dielectric barrier and the electrode, discharges the plasma into the biological surface through a formulation. The formulation may include one or more active ingredients, including but not limited to anti-oxidants, radical scavenging compounds, ultraviolet absorbing compounds, rejuvenating compounds, etc. In embodiments, the radical scavenging compound is an anhydrous, glycol-in-silicone formula with ascorbic acid and ascorbyl glucoside. In embodiments, the radical scavenging compound is a water-in-silicone emulsion with a large internal aqueous phase incorporating water-soluble active ingredients. Without being bound to theory, it is believed that the aqueous phase will form encapsulations, containing active ingredients. Rejuvenating compounds may include collagen, elastin, and the like. The formulation may include inactive ingredients, such as dyes, pigments, fragrances, essential oils, emulsifiers, viscosity modifiers, etc. In embodiments, the dye can be chemically reactive, and may respond to changes in pH induced by exposure to the plasma.

A plasma is discharged into the formulation in a container, before application to the biological surface at or near the region. Without being bound to theory, it is believed that the plasma generates beneficial species in the plasma, including ions, radicals, and long-lived RONS. The plasma treatment device may generate the plasma in proximity of the formulation, by placing the plasma treatment device near the exposed surface of the formulation while it is in the container.

In embodiments, a pre-treatment formulation enhances the effects of exposure to the plasma by including reagent compounds to generate RONS. In embodiments, a post-treatment formulation reduces the potentially harmful effects of prolonged exposure to plasma generated species. For example, the post-treatment formulation may control the pH shift of the region after exposure to plasma generated species by including buffer compounds.

A method of treatment using the plasma treatment device to generate the plasma between the plasma treatment device and the biological surface that includes at least one formulation. In embodiments, the method may include additional steps or can be practiced without all steps illustrated in the flow chart.

The method starts, and proceeds to a pre-treatment phase, including selecting a formulation, and applying the formulation to the region. As previously described, the formulation may have protective or enhancing properties that improve therapeutic results following exposure to the plasma. In embodiments, the formulation is selected for reducing exposure of the region to ultraviolet photons produced in the plasma, or for enhancing production of RONS, etc.

In embodiments, a pre-treatment formulation is applied to the region before exposure to the plasma. The method then proceeds, which includes generating the plasma. The plasma treatment device may generate the plasma in proximity to the region. The plasma treatment in block may continue until the plasma turns off. In embodiments, the method then proceeds, where a post-treatment formulation is selected. The post-treatment formulation can be applied to the region following exposure to the plasma. The pre-treatment formulation and the post-treatment formulation can be identical or different, and selected to provide different effects to the region. The method ends. In embodiments, the method includes removing the pre-treatment formulation following plasma treatment. In embodiments, the method includes removing the post-treatment formulation after applying the post-treatment formulation.

### Modular Cold Plasma Generating Device

In embodiments, the system includes a treatment device body and a head that is removeably attached to the treatment device body. The illustrated head has a mounting side facing the treatment device body and an application side carrying an electrode, and a dielectric barrier has a first side facing the electrode and a second side facing away from the electrode. The cold plasma system may include a plurality of attachable heads for cosmetic treatment over a region of a biological surface. The biological surface includes, but is not limited to, skin, hair, fingernails, etc.

In embodiments, a head is selected to produce the cold plasma to execute a particular treatment. For example, when treating a relatively small region on the biological surface, a size of plasma can be selected to avoid exposing the non-target portion of the biological surface to plasma-generated species. Here, the term “size of plasma” refers to a characteristic or a descriptive dimension of the plasma. For example, for a plasma generated by a round electrode, the characteristic dimension of the plasma is related to a diameter of the electrode.

A head can be selected and attached to the treatment device body. The head tapers from a larger size at the mounting side to a smaller size at the application side. Therefore, the head generates the plasma having a characteristic size that differs from the diameter of the mounting side of the head. While the head may have an application side that is smaller than the attachment side, it should be understood that the reverse is also possible. For example, the head may have its application side larger than the attachment side to cause a low intensity treatment over a region of the biological surface.

As illustrated, a head may include a formula reservoir and an exuding surface on the application side of the head. The exuding surface can be connected to the formula reservoir via one or more conduits. In embodiments, the formula exuding surface includes one or more nozzles on the application side of the head. In embodiments, the formula exuding surface is a porous material having a void volume to buffer the flow of formula from the formula exuding surface. The porous material may include a cured gel, a soft plastic foam, a rigid plastic foam, a natural porous material such as pumice, etc. In embodiments, the formula exuding surface may include a vent barred by one or more grills, a wire mesh screen, a patterned perforated screen, etc. In embodiments, the formula reservoir is compressed by pressure when the application side of the head is applied to the biological surface. In embodiments, the formula reservoir is compressed by a mechanism enclosed within the head including, but not limited to an electric actuator, a servo, a manually operated lever, a roller, a pair of rollers, etc. In embodiments, the formula reservoir is removable and interchangeable, and contains a prepared formula tailored to a desired therapeutic or cosmetic result.

In embodiments, the formula includes one or more cosmetic ingredients. Cosmetic ingredients may include a fragrance, a pigment, a cream, an oil, a natural extract, a moisturizer, etc. In embodiments, the formula includes one or more medicaments, for example, astringents, pharmaceutically active compounds, acid neutralizing creams, anti-oxidants, etc. In embodiments, the formula includes one or more protective compounds to protect the biological surface from potentially harmful effects of exposure to the plasma. Some non-limiting examples of such protective compounds are an anti-oxidant, a moisturizer, a clarifying cream, an acidity buffering cream, etc.

In embodiments, the head includes a flexible skirt at the application side of the head. In embodiments, the flexible skirt is made from corrugated plastic or soft rubber, and attached to the application side of the head. In embodiments, the flexible skirt is compressed by contacting the biological surface. In embodiments the flexible skirt includes a rigid spacer, restricting the compression of the skirt, thereby defining a minimum spacing between the head and the biological surface. In embodiments the flexible skirt is impermeable to gases and, when compressed, creates a contained environment for the plasma to form therein. The rigid spacer can be enclosed by the flexible skirt or can be external to it, and can be added or removed. In embodiments, the rigid spacer includes a conductive material including but not limited to a metal. In embodiments, the rigid spacer including a conductive material is biased at a voltage greater than or equal to zero. Without being bound to theory, it is believed that the rigid spacer thus biased may allow the plasma to form between the head and the rigid spacer, thereby reducing the dose of ions and electrons directed to the biological surface. In embodiments, the plasma discharging into the rigid spacer produces RONS that are contained in the volume defined by the flexible skirt.

In embodiments, the head includes a filter for filtering the plasma. The filter can be placed between the head and the biological surface, e.g., on a path of the plasma applied to the biological surface.

In embodiments, the filter is an ultraviolet filter, placed at least partially to block the path of ultraviolet photons from the plasma to the biological surface. In embodiments, the filter blocks ultraviolet photons because the filter is made of UV absorbent or UV scattering material, including, but not limited to, plastic, glass or quartz treated with a UV-blocking film, etc.

In embodiments, the filter is a chemical filter designed to sequester or convert one or more plasma generated species that would otherwise reach the biological surface. In embodiments, the filter includes a carbonaceous material, non limiting examples of which include graphene, carbon nanotubes, activated carbon paper, carbon fiber, etc. In another embodiment, the filter includes a catalytic material, non limiting examples of which include metal particles embedded in a porous matrix. In embodiments, the filter includes radical scavenging materials, for example, antioxidants, including catalases, glutathione peroxidase, superoxide dismutase (SOD), a-tocopherol (Vit. E), ascorbic acid (Vit. C), b carotene (Vit. A), selenium, etc. In embodiments, the filter includes a pH sensitive polymer that responds to changes in proton concentration by changing its porosity, surface properties, dimensions, etc. Some non-limiting examples of such pH sensitive polymers include polyacids and polybases, chitosan, hyaluronic acid, and dextran. In embodiments, the filter responds to changes in pH by opening pores and releasing one or more of the previously described radical scavenging materials.

In embodiments, the filter includes a liquid formula that is applied to the biological surface upon contact. The liquid formula may include any of the previously mentioned filter materials, carried in a liquid emulsion including but not limited to a cream or an oil. In embodiments, the liquid formula filter includes additional materials such as cosmetic ingredients, medical ingredients, etc. In embodiments, the liquid formula includes an indicator material that provides a colorimetric indicator of exposure to plasma generated species. In embodiments, the indicator material is a pH sensitive dye that will change color when the biological surface has been exposed to a concentration of plasma-generated acidifying or alkalizing species that is sufficient to alter the molecular structure of the dye. Non limiting examples of pH sensitive dye include Gentian violet, Methyl yellow, Methyl red, Cresolphthalein, Indigo carmine, etc.

In embodiments, the filter includes a charged particle filter placed between the plasma and the biological surface that attracts and neutralizes charged particles present in the plasma. In embodiments, the charged particle filter includes one or more conductive elements, individually biased at a nonzero voltage. Non-limiting examples of a conductive element include a metal screen, a metal probe, a metal ring, etc., placed near or around the dielectric material on the application side of the head. In embodiments, the charged particle filter selectively filters out positive ions by having a negative polarity, therefore neutralizing the positive ions that approach the surface of the filter. In embodiments, the charged particle filter filters out all charged particles by combining multiple conductive elements, e.g., at least one conductive element carrying a negative polarity and at least one conductive element carrying a positive polarity.

The biological surface can include contours that may affect the uniformity of exposure of the region to the plasma. Non-limiting examples of contoured biological surfaces include regions on a face and body, including but not limited to convex surfaces such as the checkbones, the chin, the eyebrows, the nose, the jaw, knuckles, ankles, elbows, knees, etc. Similarly, contoured biological surfaces may include concave surfaces, as in the area beneath the jaw, around the cars, along the neck, etc. In embodiments, a head includes a conformable material on the application side. The conformable material is configured to reversibly conform to the contours of the region. Non-limiting examples of the conformable material include gel, cured foam, rubber, plastic, etc. In embodiments, the conformable material on the head includes a consumable material, for example a dry solid, a moisturizing gel, a water soluble cream, etc.

In embodiments, the application side of the head is reversibly conformable with respect to the biological surface. In embodiments, the dielectric barrier includes a flexible surface, including but not limited to a woven dielectric cloth, such as a glass cloth, a ceramic cloth, etc. In embodiments, the electrode includes a flexible conductive surface, such as a woven metal cloth, copper mesh, stainless steel mesh, etc. In embodiments, the flexible surface included in the dielectric barrier is sealed to prevent accumulation of material abraded from the biological surface during the plasma treatment. The flexible surface can be sealed with a coating including, but not limited to, Teflon, SiOₓ film, graphene, etc.

A head may provide an air cushion between the head and the biological surface. In embodiments, the head includes a plurality of air conduits that at least partially surround the electrode and the dielectric barrier. In operation, the air mover provides air to the air conduits (e.g., nozzles, vents, etc.) that direct a vectored flow of air away from the head. The flow of air may create an air cushion that prevents or at least minimizes a contact between the head and the biological surface. In embodiments, the air mover is an electric fan, located within the head. The air mover may operate independently from the electrode and can be turned on and turned off without altering the state of the plasma.

Cold Plasma Device with Sensors

In embodiments, the cold plasma treatment device includes one or more sensors to measure plasma parameters. Based on the measured plasma parameters, a controller may control the cold atmospheric plasma and maintain a predetermined cosmetic treatment over a region of a biological surface.

As previously described, in embodiments, the cold atmospheric plasma is formed using the biological surface as a floating reference electrode. Without being bound to theory, it is believed that such an arrangement is sensitive to non-uniform distribution of water and ion concentrations over the biological surface. It is believed that a localized region that is relatively rich in ions, such as a sweat gland, may provide a preferred conductive path for plasma-generated charged species, and the cold atmospheric plasma may form preferentially at such a site on the biological surface. In turn, plasma preference for a particular location over another on a biological surface introduces poorly controlled non-uniformity in treatment and variability in plasma dosage over the region treated by the plasma. It is believed that uniformity is an important criterion in the operation of a cold atmospheric plasma source. Therefore, in embodiments, the design of the plasma treatment device takes into account the sensitivity of the cold atmospheric plasma to variations in properties of the surface.

Uniformity of the plasma is defined in terms of a variability of one or more plasma parameters, for example, discharge power, discharge volume, the concentrations of plasma generated species, etc. In a highly variable system, for example, where the treatment region contains many discrete sub-regions of disparate properties, the plasma treatment device may exhibit discontinuities in the discharge current or discharge voltage as the plasma treatment device translates between ion-rich and ion-poor sub-regions of the surface. Without being bound to theory, it is believed that a plasma source, passing over a conductive sub-region may exhibit a spike in discharge current and a corresponding drop in discharge voltage.

In embodiments, the controller actuates an electronic ballast circuit, connected to the electrode. Without being bound to theory, it is believed that an electronic ballast circuit may permit the controller to regulate the current to electrode, thereby preventing thermal runaway of the plasma and constriction of the plasma at one or more localized spots on the biological surface.

The plasma source incorporates one or more sensors to measure parameters of a cold atmospheric plasma and a biological surface. In embodiments, the plasma treatment device includes sensors that measure plasma parameters. The plasma parameters may include measurements of the electric current discharged into the biological surface, the voltage drop between the dielectric barrier and the surface. The plasma parameters may include one or more parameters indicative of the energy density of the plasma, such as the spectrum of light emitted by the plasma, the ion-density in the plasma, or variation in time of the prior-mentioned parameters that would indicate non-uniform surface treatment. Without being bound to theory, it is believed that one or more short-lived discontinuities in the discharge voltage or discharge current indicates a non-uniformity in the form of preference of the cold atmospheric plasma for one or more highly localized ion-rich regions on the surface.

In embodiments, one or more sensors, placed on the surface at or near the treatment region, measure parameters of the plasma or of the biological surface. For example, the plasma treatment device may include ion sensors, such as pH sensors or chloride sensors, light sensors, reactive oxygen sensors, a surface temperature sensor, a distance sensor, humidity sensors, etc.

In embodiments, sensors placed either on the surface or on the plasma treatment device measure the ambient environment. Such sensors may include ion sensors, light sensors, reactive oxygen sensors, temperature sensors, humidity sensors, etc.

In embodiments, a position reference sensor placed on the plasma treatment device is operably coupled to a distance sensor on the biological surface. The position reference sensor may determine the distance of the dielectric barrier from the surface. In embodiments, a distance sensor, such as a laser rangefinder included in the plasma treatment device, measures the distance from the dielectric barrier to the surface.

In embodiments, the sensors communicate with the controller, as part of the plasma source. The controller can be operably coupled to the plasma treatment device, and may receive input from the sensors and process that input to determine control data for the plasma treatment device. In embodiments, the control data includes, but is not limited to, signals sent to electronic components of the plasma treatment device to modulate the current or the voltage provided to the electrode, and signals sent to other components of the plasma treatment device to produce a perceptible signal. In embodiments, the perceptible signal is a haptic feedback or an audible or visible indicator. In embodiments, the controller sends control data in response to an unsafe dose of energy or reactive species produced by the plasma.

As previously described, without being bound to theory, a plasma dose is believed to determine exposure to one or more plasma generated species such as, reactive chemical species, energetic species including ions and electrons, photons, etc.

In embodiments, a plasma dose is a concentration of a given species imparted to a given region on the biological surface over a period of time, expressed as a number per unit-area, per unit-time (such as “per square-centimeter seconds”). In embodiments, the controller determines a treatment duration and control data to send to the plasma treatment device, by integrating the plasma dose over the area of the dielectric barrier, to provide a plasma dose per unit time. In embodiments, when the plasma treatment device remains over a given region on the biological surface for a length of time such that the plasma is likely to harm the surface, the plasma treatment is considered unsafe. Conversely, in embodiments, if the treatment device remains over the given region for a length of time such that the plasma is unlikely to have the desired effect, the plasma treatment is considered to have provided an ineffective dose. In embodiments, these doses are not unique values, but rather are thought to occur in ranges. As such, a controller may determine an unsafe range or an ineffective range of doses, wherein it will send control data to the plasma treatment device to produce a perceptible signal or to modulate the plasma, or both.

In embodiments, the controller responds to an unsafe dose by sending a signal for the source to be moved away from the region on the biological surface toward a second region. The controller may respond to an unsafe dose by sending control data to the electronic components of the plasma treatment device to turn off the plasma, or to modulate the power provided to the electrode to diminish the generation of energetic species and reactive species in the plasma.

In embodiments the plasma is generated by a plurality of pixelated electrodes arranged in a matrix. The pixelated electrodes can be individually addressable by the controller, where the controller determines a discharge power for a given pixelated electrode. In embodiments, the controller uses input from current and voltage sensors for the pixelated electrodes to counteract non-uniform plasma constriction or localization. In embodiments, when the plasma localizes to a spot on the biological surface having disparate chemical or physical properties, the controller receives input indicating which pixelated electrodes are drawing a disproportionate rate of electrical power, relative to the average for the matrix. The controller may modulate the plasma by turning off the electrodes that are drawing excess power, thereby distributing plasma energy to operational electrodes O, and diminishing the undesirable effects of plasma non-uniformity near the non-operational electrodes NO.

The components of the cold plasma system may communicate directly through wired and powered connections. These components may communicate to each other via a network (not shown), which may include suitable communication technology including, but not limited to, wired technologies such as DSL, Ethernet, fiber optic, USB, and Firewire; wireless technologies such as WiFi, WiMAX, 3G, 4G, LTE, and Bluetooth; and the Internet.

In embodiments, the controller includes a non-transitory computer readable medium having computer executable instructions and data stored thereon that cause, in response to execution by one or more processors of a computing device, the computing device to implement a method of treatment as described herein.

In embodiments, the method of treatment of the region of the biological surface with the cold atmospheric plasma includes generating the cold plasma between the plasma treatment device and the region. The method of treatment may include measuring one or more treatment parameters with one or more sensors and determining a plasma dose from the treatment parameters. In embodiments, the method of treatment includes modulating one or more of the treatment parameters to adjust the plasma dose, and switching off the cold atmospheric plasma.

In embodiments, the method may include additional steps or can be practiced without all steps illustrated in the flow chart. The method starts at block, and proceeds to block where one or more sensors measure treatment parameters, for example, ambient parameters and surface parameters. In embodiments, prior to generating the plasma, the method includes placing at least one sensor onto the biological surface at or near the region. As previously described, the sensors can be operably coupled to the controller, and may provide sensor input to the controller to be used in block to determine plasma parameters necessary for effective treatment. In embodiments, the plasma parameters are defined by default values, and the controller does not act until the plasma has been turned on. In embodiments, the plasma parameters include a discharge voltage as a function of time, a discharge current as a function of time, a plasma temperature as a function of time, or a gas temperature near the region as a function of time. In embodiments, the sensor measurements are provided to a data storage system, which may aggregate the measurements with other sensor data. In embodiments, a parameter engine communicates parameter information to the controller. The parameter engine determines a treatment dose based on aggregate sensor inputs accumulated and stored in a data storage system, and further determines a set of plasma parameters that are provided to the controller.

A cosmetic formulation is applied to the treatment region. In embodiments, the cosmetic formulation enhances plasma treatment. In embodiments, the cosmetic formulation protects the biological surface from harmful aspects of the plasma. A formulation engine may determine the formulation, which may receive input from the data storage system. In embodiments, the formulation engine applies machine learning to optimize the components of the formulation for a given purpose such as radical scavenging, UV absorption, electrical conductivity, thermal conductivity, etc.

The plasma treatment device applies the cold atmospheric plasma to the biological surface at the treatment region. A post-plasma formulation is applied to the treatment region of the biological surface. The formulation can be determined by a formulation engine. In embodiments, the post-plasma formulation can be the same as the formulation. In embodiments, the post-plasma formulation can be different from the formulation. In embodiments, the post-plasma formulation neutralizes ions and moisturizes the biological surface. In embodiments, the post-plasma formulation counteracts possible oxidative effects of plasma treatment by including anti-oxidant ingredients.

The treatment can be repeated. In embodiments, the controller determines whether the treatment dose has been met. Where the treatment dose has not been met, the controller may repeat the sensor measurements, determine new plasma parameters, and modulate the plasma to provide an effective and safe dose of plasma generated species. In embodiments, the treatment is not repeated, and the method ends.

The controller may determine plasma parameters from a group including a current provided to the electrode, a driving frequency, a voltage waveform, a peak to peak voltage, a root mean square voltage, a plasma temperature, a gas temperature, optical emission from the plasma, etc.

In embodiments, the controller determines an indicator of uniformity of the cold atmospheric plasma. As previously described, uniformity describes the spatial distribution of plasma between the second side of the dielectric barrier and the biological surface, as well as whether a time-averaged flow of current between the two surfaces is evenly spread across the treated region on the biological surface. In embodiments, the controller sends control data to the plasma treatment device to modulate one or more of the plasma parameters in response to changes in the indicator of uniformity. The controller may determine the indicator of uniformity intermittently, based on sensor inputs provided to the controller.

As understood by one of ordinary skill in the art, a “data storage system” as described herein can be any suitable device configured to store data for access by a computing device. An example of the data storage system is a high-speed relational database management system (DBMS) executing on one or more computing devices and being accessible over a high-speed network. However, other suitable storage techniques and/or devices capable of providing the stored data in response to queries can be used, and the computing device can be accessible locally instead of over a network, or can be provided as a cloud-based service. The cloud storage system may also include data stored in an organized manner on a computer-readable storage medium.

In general, the word “engine,” as used herein, refers to logic software and algorithms embodied in hardware or software instructions, which can be written in a programming language, such as C, C++, COBOL, JAVA™, PHP, Perl, HTML, CSS, JavaScript, VBScript, ASPX, Microsoft .NET™, PYTHON, and/or the like. An engine can be compiled into executable programs or written in interpreted programming languages. Software engines can be callable from other engines or from themselves. Generally, the engines described herein refer to logical modules that can be merged with other engines, or can be divided into sub engines. The engines can be stored in any type of computer readable medium or computer storage device and be stored on and executed by one or more general purpose computers, thus creating a special purpose computer configured to provide the engine or the functionality thereof.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, application specific integrated circuit (ASIC), controller or data processor that is specifically programmed, configured or constructed to perform one or more of the computer-executable instructions described above. Of course, any logic or algorithm described herein can be implemented in software or hardware, or a combination of software and hardware.

From the foregoing, it will be appreciated that specific embodiments of the technology have been described herein for purposes of illustration, but that various modifications can be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Where methods are described, the methods may include more, fewer, or other steps.

Additionally, steps can be performed in any suitable order. Accordingly, the disclosure can encompass other embodiments not expressly shown or described herein.

For the purposes of the present disclosure, lists of two or more elements of the form, for example, “at least one of A, B, and C,” is intended to mean (A), (B), (C), (A and B), (A and C), (B and C), or (A, B, and C), and further includes all similar permutations when any other quantity of elements is listed.

### Terminology

The present disclosure may reference quantities and numbers. Unless specifically stated, such quantities and numbers are not to be considered restrictive, but representative of the possible quantities or numbers associated with the present disclosure. Also, in this regard, the present disclosure may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices or components such as a processor (e.g., a microprocessor), a central processing unit (CPU), a digital signal processor (DSP), an application-specific integrated circuit (ASIC), a field-programmable gate array (FPGA), or the like, or any combination thereof, and can include discrete digital or analog circuit elements or electronics, or combinations thereof.

An embodiment includes one or more data stores that, for example, stores instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a device and/or system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein can be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of example embodiments of the present disclosure. It will be apparent to one skilled in the art, however, that the embodiments disclosed herein can be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present disclosure may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The principles, example embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A modular mask system configured for cosmetic analysis and treatment, the modular mask system comprising: a wearable modular mask, comprising: a front portion attached to a rear portion that comprises a grid system configured for a cosmetic analysis, a cosmetic treatment, or both; a jig configured to accept and guide a printer device for application of a cosmetic style to a portion of skin of the individual that is adjacent to the jig; and an attachment site configured to accept and position a therapy device for application of a cosmetic treatment to a portion of skin of the individual that is adjacent to the attachment site.

Embodiment 2. The modular mask system of Embodiment 1 or any other Embodiment, further comprising the printer device.

Embodiment 3. The modular mask system of Embodiment 1 or any other Embodiment, further comprising the therapy device.

Embodiment 4. The modular mask system of Embodiment 1 or any other Embodiment, further comprising control circuitry configured to control one or more cosmetic analyses and/or treatments.

Embodiment 5. The modular mask system of Embodiment 4 or any other Embodiment, further comprising a computational device comprising circuitry configured to interact with the control circuitry for coordination, observation, or management of cosmetic analysis and treatment by the computational device.

Embodiment 6. The modular mask system of Embodiment 5 or any other Embodiment, wherein the computational device comprises a smart phone, a tablet, a laptop computer, a desktop computer, a smart watch, a wearable computational device, or any combination thereof.

Embodiment 7. The modular mask system of Embodiment 1 or any other Embodiment, wherein the wearable modular mask comprises a lower detachment point at which an eye portion of the wearable modular mask is detachable from a mouth portion of the wearable modular mask.

Embodiment 8. The modular mask system of Embodiment 1 or any other Embodiment, further comprising a scalp portion of the wearable modular mask, wherein a rear portion of the scalp portion comprises a grid system configured for a cosmetic analysis, a cosmetic treatment, or both.

Embodiment 9. The modular mask system of Embodiment 8 or any other Embodiment, wherein the wearable modular mask comprises an upper detachment point at which the scalp portion of the wearable modular mask is detachable from an eye portion of the wearable modular mask.

Embodiment 10. The modular mask system of any one of Embodiments 1-9 or any other Embodiment, wherein the modular mask system comprises circuitry configured for a cosmetic analysis of a portion of skin of the individual based on a feature of the portion of skin.

Embodiment 11. The modular mask system of any one of Embodiments 1-9 or any other Embodiment, wherein the modular mask system comprises circuitry configured to interact with a computational device that comprises circuitry configured for a cosmetic analysis of a portion of skin of the individual based on a feature of the portion of skin.

Embodiment 12. The modular mask system of any one of Embodiments 1-11 or any other Embodiment, wherein the grid system is configured for an optical characterization of a portion of skin of the individual, an electrical characterization of a portion of skin of the individual, a light therapy, a micro-current treatment, a radio-frequency (RF) warming treatment, a cold plasma treatment, an acoustic energy treatment, or any combination thereof.

Embodiment 13. The wearable modular mask system of any one of Embodiments 1-12 or any other Embodiment, wherein the printer device is configured for precise application of a makeup to an eyebrow portion of the face of the individual.

Embodiment 14. The wearable modular mask system of any one of Embodiments 1-13 or any other Embodiment, further comprising an aperture configured to accept at least part of a smartphone camera lens thereto for smartphone-facilitated imagery of at least a portion of the face of the individual with placement of the wearable modular mask thereto via camera circuitry of the smartphone.

Embodiment 15. The wearable modular mask system of any one of Embodiments 1-14 or any other Embodiment, wherein the attachment site is positioned at a cheek portion of the wearable modular mask.

While illustrative embodiments have been illustrated and described, it will be appreciated that various changes can be made therein without departing from the spirit and scope of the invention.

The embodiments of the invention in which an exclusive property or privilege is claimed are defined as follows:

1. A modular mask system configured for cosmetic analysis and treatment, the modular mask system comprising:
a wearable modular mask, comprising:
a front portion attached to a rear portion that comprises a grid system configured for a cosmetic analysis, a cosmetic treatment, or both;
a jig configured to accept and guide a printer device for application of a cosmetic style to a portion of skin of the individual that is adjacent to the jig; and
an attachment site configured to accept and position a therapy device for application of a cosmetic treatment to a portion of skin of the individual that is adjacent to the attachment site.

2. The modular mask system of claim 1, further comprising the printer device.

3. The modular mask system of claim 1, further comprising the therapy device.

4. The modular mask system of claim 1, further comprising control circuitry configured to control one or more cosmetic analyses and/or treatments.

5. The modular mask system of claim 4, further comprising a computational device comprising circuitry configured to interact with the control circuitry for coordination, observation, or management of cosmetic analysis and treatment by the computational device.

6. The modular mask system of claim 5, wherein the computational device comprises a smart phone, a tablet, a laptop computer, a desktop computer, a smart watch, a wearable computational device, or any combination thereof.

7. The modular mask system of claim 1, wherein the wearable modular mask comprises a lower detachment point at which an eye portion of the wearable modular mask is detachable from a mouth portion of the wearable modular mask.

8. The modular mask system of claim 1, further comprising a scalp portion of the wearable modular mask, wherein a rear portion of the scalp portion comprises a grid system configured for a cosmetic analysis, a cosmetic treatment, or both.

9. The modular mask system of claim 8, wherein the wearable modular mask comprises an upper detachment point at which the scalp portion of the wearable modular mask is detachable from an eye portion of the wearable modular mask.

10. The modular mask system of claim 1, wherein the modular mask system comprises circuitry configured for a cosmetic analysis of a portion of skin of the individual based on a feature of the portion of skin.

11. The modular mask system of claim 1, wherein the modular mask system comprises circuitry configured to interact with a computational device that comprises circuitry configured for a cosmetic analysis of a portion of skin of the individual based on a feature of the portion of skin.

12. The modular mask system of claim 1, wherein the grid system is configured for an optical characterization of a portion of skin of the individual, an electrical characterization of a portion of skin of the individual, a light therapy, a micro-current treatment, a radio-frequency (RF) warming treatment, a cold plasma treatment, an acoustic energy treatment, or any combination thereof.

13. The wearable modular mask system of claim 1, wherein the printer device is configured for precise application of a makeup to an eyebrow portion of the face of the individual.

14. The wearable modular mask system of claim 1, further comprising an aperture configured to accept at least part of a smartphone camera lens thereto for smartphone-facilitated imagery of at least a portion of the face of the individual with placement of the wearable modular mask thereto via camera circuitry of the smartphone.

15. The wearable modular mask system of claim 1, wherein the attachment site is positioned at a check portion of the wearable modular mask.
